SEQUENCE LISTING (66 sequences)

SEQ ID NO: 1 - DNA, 414 bp, Homo Sapiens
ggcggctcggggcccagcgcggggtccgggggaggcggcttcgggggttcggcggcggtggcggcggcgacggcttcgggcggcaaatccggcggcgggagctgtggagggggtggcagttactcggcctcctcctcctcctccgcggcggcagcggcgggggctgcggtgttaccggtgaagaagccgaaaatggagcacgtccaggctgaccacgagcttttcctccaggcctttgagaagccaacacagatctatagatttcttcgaactcggaatctcatagcaccaatatttttgcacagaactcttacttacatgtctcatcgaaactccagaacaaacatcaaaaggaaaacatttaaagttgatgatatgttatcaaaagtagagaaaatgaaaggagagcaagaa

SEQ ID NO: 2 - DNA, 401 bp, Homo Sapiens
acaggtgaaaaaccaaatactttctagggatgaccttgatgacataattcagtcatctcaaacagtctcagaggacggtgactcgctttgctgtaattgtaagaatgtcatattactcattgatcaacatgaaatgaagtgtaaagattgtgttcacctattgaaaattaaaaagcattttgtttatgtaaaagattaacagaacttaaagataatcactgtgagcaacttagagtaaaaattcgaaaactgaaaaataaggctagtgtactacaaaagagactatctgaaaaagaagaaataaaatcgcagttaaagcatgaaacacttgaattggaaaaagaactctgtagtttgagatttggcctacagcaagaaaaaaagaaaagaagaaatgttga

SEQ ID NO: 3 - DNA, 373 bp, Homo Sapiens
gagagtttgtggaagatggcgcctgttgtgacagggaaatttggtgagcggcctccacctaaacgacttactagggaagctatgcgaaattatttaaaagagcgaggggatcaaacagtacttattcttcatgcaaaagttgcacagaagtcatatggaaatgaaaaaaggtttttttgcccacctccttgtgtatatcttatgggcagtggatggaagaaaaaaaaagaacaaatggaacgcgatggttgttctgaacaagagtctcaaccgtgtgcatttattgggataggaaatagtgaccaagaaatgcagcagctaaacttggaaggaaagactattgcacagccaaaacattgtatatatctgacta

SEQ ID NO: 4 - DNA, 394 bp, Homo Sapiens
gattcggagaatgatggagacatttcagcaacttattacttataaagtcataagcaatgaatttaacagtcgaaatctagtgaatgatgatgatgccattgttgctgcttcgaagtgcttgaaaatggtttactatgcaaatgtagtgggaggggaagtggacacaaatcacaatgaagaagatgatgaagagcccatccctgagtccagcgagctgacacttcagggaacttttgggagaagaaagaagaaacaagaaaggttcctcgagtggaccccctggaaactgaacttggtgttaaaaccctggattgtcgaaaaccacttatcccttttgaagagtttattaatgaaccactgaatgaggttctagaaatggataagatttactttt

SEQ ID NO: 5 - DNA, 408 bp, Homo Sapiens
ggaatatgtcttacccctgactgtgaaggtgtcatttctaagattatcatcttcagcagtggtggtgaagttaaatgtgaatttgaacacaaggtcataaaagaaaaggttcctccaagacctattctgaaacagaaatgttctagcctagagaaactaagactgaaagaagacaaaaaattgaagagaaagatccaaaaaaaagaagcaaaaaagttagcacaagaaagaatggaggaggacttaagagaaagtaatccacccaaaaatgaagacagaaagaaactgtagacaatgttcaagcgttgtcagttccttgatgacagaattctacagtgtataaagcagtatgcttgacaggattaaatccggcatacagaatacagccatgcttctaaagaattgttt

SEQ ID NO: 6 - DNA, 387 bp, Homo Sapiens
gggaagcagaaggatttggagtttctttttaaagtgattccttcctttcccctttcatttttccactgtgggtgttattatcctgacaatttgtcatacatttcctgtctttaaaaaataactgtatactaagcaaaactcaggtcttaaaaataaatatgaatttagattccatacatcgattaattgaggaaacacagatcttccagatgcaacaatcatcaattaagtcacgcggcgacatggtggcccctgcctcaccccccagggatacctgtaatacctgcttcccacttcatgggctacaatctcatgctgtcacaatttctgtgctcactcatataacaccacaaatgggatatttgtgaagaacttcgctgcggagct

SEQ ID NO: 7 - DNA, 407 bp, Homo Sapiens
agggacagctcttgcatcgagaccccttcactgtcatctgtggccgaaagaagtgccttcgccatgtctttctcttcgagcatctcctcctgttcagcaagctcaagggccctgaaggggggtcagagatgtttgtttacaagcaggcctttaagactgctgatatggggctgacagaaaacatcggggacagcggactctgctttgagttgtggtttcggcggcggcgtgcacgagaggcatacactctgcaggcaacctcaccagagatcaaactcaagtggacaagttctattgcccagctgctgtggagacaggcaagcccacaacaaggagctccgagtgcagcagatggtgtcatggcattgggaataaacccttctggacataaagcccttggggagcga

SEQ ID NO: 8 - DNA, 399 bp, Homo Sapiens
gcggcggcggcccctcgcagcagctggccggcgggcccccccagcagttcgcgctctccaactccgcggccatccgggccgagatccagcgcttcgagtccgtgcatcccaatatctacgccatctacgacctgatcgagcgcatcgaggatttggcgctgcagaaccagatccgggagcacgtcatctccatcgaggactcgtttgtgaacagccaggagtggacgctgagccgctccgtaccggagcttaaagtgggcatagtggggaacctgtctagcgggaagtcaagccctggtgcaccgctatctgacggggacctatgtccaggaggagtcccctgaaggggggcggtttaagaaggagattgtggtggatggcagagttcctgctgtgatc

SEQ ID NO: 9 - DNA, 402 bp, Homo Sapiens
ggctggcagtagaggtgaccgaggcggtggcggcggaggcggcaccgattgctgtgtcggccccagtgcggccgaagtcgcggtagagcgtagccccacgcccctcccccgtccgcgccctccctctttccctggggatggagaaggcgacggttcctggtggcggcggcgacggcttgcagaaggagaagggagccccccggcggtggcggcttgtggcgggcccccccgcggcggcggaggtcggcggcggcgttggcggcagcagcagagctcgctcggcctcgtctcctcgtgggatggtgcgagtctgcgacctgctcctgaagaagaagccgccgcagcagcagcaccacaaggccaagcgtaaccggacttgccaccccccagcagcagcgaaac

SEQ ID NO: 10 - DNA, 393 bp, Homo Sapiens
gcggcggcggcccctcgcagcagctggccggcgggcccccccagcagttcgcgctctccaactccgcggccatccgggccgagatccagcgcttcgagtccgtgcatcccaatatctacgccatctacgacctgatcgagcgcatcgaggatttggcgctgcagaaccagatccgggagcacgtcatctccatcgaggactcgtttgtgaacagccaggagtggacgcttgagccgctccgtaccggagcttaaagtgggcatagtggggaacctgtctagcgggaagtcagccctggtgcaccgctatctgacggggacctatgtccaggaggagtccctgaaggggggcggttaagaaggagattgtggtggatggcagagttcctgctgc

SEQ ID NO: 11 - DNA, 402 bp, Homo Sapiens
attatgaagtaactgaacttttggtcaagcatggtgcctgtgtaaatgcaatggacttgtggcaattcactcctcttcatgaggcagcttctaagaacagggttgaagtatgttctcttctcttaagttatggtgcagacccaacactgctcaattgtcacaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctagaaaagc

SEQ ID NO: 12 - DNA, 400 bp, Homo Sapiens
cctcagcatacccaccgagcagctgccagcctgggctgagggtgggcatgaggcaggagtcagcacttggacctagggatgtgaggttttctgtgccccaagtttgtgggaaggtgggcactactgctgggcccacagacacagccagctggcaaaagggaggtctagcccagcagagagatgaggacattttgcttctccttcatgcccacagcatgagctgagcttctgctttgctggaaatgaaataaacttggtatgaattgtgccaaggcctccccagttgtcatcctgcctcttgttgccctcccttgtccttgccccccaccccacacccatgcccctgtttccttacagattttgatattgtctaatgtgtaatagaaccagccgagtccca

SEQ ID NO: 13 - DNA, 84 bp, Homo Sapiens
cttacctcatttctgaatgtgcatttccagccttcttgctctcagagctattgttcaagcagaaaacaagctgcttttattaca

SEQ ID NO: 14 - DNA, 104 bp, Homo Sapiens
gagagaactagtctcgagttttttttttattcttctatattctatgaatatggtgctgtcctgtcatttaattattataatatatgtgaactgctggaggtaaa

SEQ ID NO: 15 - DNA, 410 bp, Homo Sapiens
tcgatccttagtgacttaacaatcaggccttaattgaaacacacacacacattgttattgacagtgtagaaatactgactcatagaaaaattcacccatatttagttagcagactaacaggaacagcagcagcagcagcagctggtcatgcttctgtgtgttgctagcaacaagaaaccatgacagcaaggccccaaacaggaacctcctgcattttctcatctgtgatgaggcacacttgatgctggggattaatgagcctgaagatataaagcagtgtttaccactggaaaatgtctcctacactaaaagcagaggtaagtatcaatgcaaaccgagtgcagctataaagccttgatttctctggaaattatgtacaaactaatacaaataatctcattacttgaaac

SEQ ID NO: 16 - DNA, 380 bp, Homo Sapiens
gctacggctgctccggagctggtggcgccgcgataggagagccgatggccaagtggggtgagggagacccacgctggatcgtggaggagcgggcggacgccaccaacgtcaacaactggcactggacggagagagatgcttcaaattggtccacggataagctgaaaacactgttcttggcagtgcaggttcaaaatgaagaagtcaagtgtgaggtgacggaagtgagtaagcttgatggagagtcatccattaacaatcgcaaagggaaacttatcttcttttatgaatggagcgtcaaactaaactggacaggtacttctaagtcaggagtacagtacaaaggacatgaggagatccccaatttgtctgatgaaaac

SEQ ID NO: 17 - DNA, 117 bp, Homo Sapiens
cattaccttacagtgtaaacaggagtctaatttgtatcaatactatgttttggttgtaatattcagttcactcacccaatgtacaaccaatgaaataaaagaagcatttaaaaggaa

SEQ ID NO: 18 - DNA, 404 bp, Homo Sapiens
  misc_feature (1)...(404): n = g, a, t, or c
ggcgtgtgggtctcgcagcgttgctcacagaacagagtagaggcggcggcggcggcggccggacccagactggtagtgaggcgttggaccccgagccgctgcaatgccgctggagctggagctgtgtcccgggcgctgggtgggcgggcaacacccgtgcttcatcattgncgagatcggccagaaccaccagggcgacctggacgtagccaagcgcatgatccgcatggccaaggagtgtggggctgattgtgccaagttccagaagagtgagctagaattcaagtttaatcggaaagccttggacaggccatacacctcgaagcattcctgggggaagacgtacggggagcacaaacgacatctggagttcagccatgaccagtcagggagctgagaggtcc

SEQ ID NO: 19 - DNA, 387 bp, Homo Sapiens
gacggaccgagaccggagatgttttcaagcccggctccggcggctttacaggcggctgcagcggcgacgaagacaacgacagcgacggctacgccgaagcactcgaaccgggggtgaagcctcctgcgccggccttgcctcggatccaggatgagaagactgataaaagaagaagctagctgaacagctgtaaaatgcccaaatctgggttcacaaaaccaattcagagtgaaaattctgacagtgacagcaatatggtagagaaaccatatggaagaaagagtaaagacaagattgcatcctacagcaaaactccaaaaattgaacgaagtgatgtgagcaaggagatgaaagagaaatcatccatgaaaccgtaaacttcctttc

SEQ ID NO: 20 - DNA, 405 bp, Homo Sapiens
gcaggaggggccttgccagcttccgccgccgcgtcgtttcaggacccggacggcggattcgcgctgcctccgccgccgcggggcagccggggggcagggagcccagcgaggggcgcgcgtgggcgcggccatgggactgcgccggatccggtgacagcagggagccaagcggccgggccctgagcgcgtcttctccggggggcctcgccctcctgctcgcggggccggggctcctgctccggttgctggcgctgttgctggctgtggcggcggccaggatcatgtcgggtcgccgctgcgccggcgggggagcggctgcgcgagcgccgcggccgaggccgtggagccggccgccgaagctgttcgaggcgtgccgaacggggacgtggaacgagtaagaggctg

SEQ ID NO: 21 - DNA, 634 bp, Homo Sapiens
  misc_feature (1)...(634): n = g, a, t, or c
gccaactcagtccagcagaacaaaatgtagctgccattcttggagtctctgaaagctttattgggaagaaagcatcaggccaagccatcggaaagaaggtggacaagaacgttgtcaacaggctatatctgtcttttgttctttataccttgctcaaagagaccaacatttggactgtatctgaaaaatttaatatgcctcgaggatatatacaaaatcttctcactggaactgcctcattctcatcttgtgtgttacatttctgtgaggagcttgagggagttttgggtttacagagcccttttggtagaacttaccaagaagctgactactgtgtaaagggcagaattaatccctctatgggaagttctnggagttttagagggtcgagcaaaacagtttttcagngccnggtaccaaaagtctaatgccttagctaagcaaaccctgaangnttctanggncaattggtcnttttttaagaccccaagccagcaaattgtttatncaaaaatctnttcntnaaaaccaaacctcaaaanggnnaaaagtccnaaatgcttttnttcccgggggngggggttnttcccggcaaacngaantttttgngggaanttttttttaatttttttng

SEQ ID NO: 22 - DNA, 648 bp, Homo Sapiens
  misc_feature (1)...(648): n = g, a, t, or c
gggaggcggcggcagcgttaagtgagaaaggaaaaaagacaacgaggaaaaaggaggtgtccgggtagggcaacgcggcgacacccgaggcctggtggtggcggcggatcgagatattcaaggctgaagcagctacggaacggcagcggcggcggtcggacaaactgactgaccgagccgggtggtggcgggagcagcgggagcagccggaacgatgccggccgtgagcctcccgcccaaggagaatgcgctcttcaagcggatcttgaggtgttatgaacataaacagtatagaaatggattgaaattctgtaaacaaatactttctaatcccaaatttgcagagcatggagaaaccttggctatgaaaggattaacattgaactgtttggggaaaaaggaagaacttatgaattggttcctagaggtttgagaaatgacttgaagagtcatgtgtgttggccacgtttatggcctttttcaaggtcanacaagaagtntgatgaannccttaantgttacagaaatgcctaaatgggataagacatcttaaattttaagggnctttcttctacaantcaatccaaactngnggnttccnggaaccaggtttnanttcttcanttncncctcccaaagcattatgnt

SEQ ID NO: 23 - DNA, 639 bp, Homo Sapiens
  misc_feature (1)...(639): n = g, a, t, or c
cggtggcggcggaggcggcaccgattgctgtgtcggccccagtgcggccgaagtcgcggtagagcgtagccccacgcccctcccccgtccgcgccctccctctttccctggggatggagaaggcgacggttccggtggcggcggcgacggctgcagaaggagaagggagccccccggcggtggcggctgtggcgggcccccccgcggcggcggaggtcggcggcggcgttggcggcagcagcagagctcgctcggcctcgtctcctcgtgggatggtgcgagtctgcgacctgctcctgaagaagaagccgccgcagcagcagcaccacaaggccaagcgtaaccggacttgccgaccccccagcagcagcgaaagcagcagcgacagcgacaacagcggcggcggtggaggcggcggtggagcggaagtggcggcggcggcaccagcantaacaacagcgaggaaanaaaggacacacaccaggaanagaggttntgaggggagttttatttggntcagattattggaaantcaancttgnaaacttccaggtnntctataangtcnnttgtngngcatacntangaantannccaaaannagntttnatgggagttttacnaaacncagtttggatc

SEQ ID NO: 24 - DNA, 663 bp, Homo Sapiens
  misc_feature (1)...(663): n = g, a, t, or c
ctnngtttttttttttttttttttccagactcttctgttcttttatatctcagaaaggattgggttttcaggttgcaaaatcttttccagctctgcataggtaggtagcatctcactgaggaatggagtatttaccacctattgttctgtnccagtctagtagagctttagcaaaanctacaggcaacaaattctatttttaacatcctgttacacaaacaaatatgctgagtatgcacacaaataaatggtgaaagaggcncaaagaagtgaaaacaatcgtgcatggtaggaatatttgaattgtnttacatgtcctttaatattgntttaacagtnatatttttacattttcaattggaatgaaaagcatgtctgtgttcgaataatttttcatcgnncnctcatttttttgattcccnanctaatgagnagaaancagtgatgattgcaaaatgtttcccnccctnaaggaatncncgtnngaattcttgcagntcctggaganctccntantttangncntatataggtanngatctatactccctcggggggtcttagcctnncgcnncctnccttccntctcacnancattgttntctanngcnnctcanntaantnctncaggcccncaantgnntatnnanccncnnncntntc

SEQ ID NO: 25 - DNA, 638 bp, Homo Sapiens
  misc_feature (1)...(638): n = g, a, t, or c
cccggaacctgcaaggcctggtctgggacccacacaaccgtaggagacaggtcctgaatacccgggcccaagagcccaagctgtgctggcctcagggtttctcctggagtcaccgagccgtggtccacttcgtcgtgcctgtgaagaaccaggcacgctgggtacagcaattcatcaaagacatggaaaacctgttccaggtcaccggtgacccacacttcaacatcgtcatcactgactatagcagtgaggacatggatgttgagatggcactgaagaggtccaagctgcggagctaccagtacgtgaagctaagtggaaactttgaacgctcagctggacttcaggctggcatagacctcgtgaaggacccgcacagcatcatcttcctctgtgacctccacatcacttcccacttggagtcatngatgccattcggaacacttgtgtggagggaaaagaagggcttttgccccctggtgataaggttgnnttgggggcncccccaanggctgaggctcgggagggaaaagggttgggnnttggatttacaatttncctganangatgggggcntaaccaaaaggantccaaancctgggngggaaaaanggnacttttnnaggaatttcaangcn

SEQ ID NO: 26 - DNA, 90 bp, Homo Sapiens
gtgagatgaatgttcccccttcaattctccttatttgccaaatattttcatttccttttgtcattatagaaaataaaaccatgcatcaca

SEQ ID NO: 27 - DNA, 85 bp, Homo Sapiens
aggaaccaaagaagacatggtccctgtcctcatggttcagacagggaggcagacattaaacaactaattatcagttattcaatta

SEQ ID NO: 28 - DNA, 638 bp, Homo Sapiens
  misc_feature (1)...(638): n = g, a, t, or c
gcgactcggggacctggagctgacgcctagacacttgtattagctttaatagaagagaaatggaggagccatagaatattaaggatgaattcaggaaggcctgagaccatggaaaacttgcctgctctctacactattttccaaggagaggttgctatggtgacagactatggggcctttatcaaaatcccaggctgtcggaagcaaggtctggtccatcgaactcatatgtcatcctgtcgggtggataagccctctgagatagtagatgttggagataaagtgtgggtgaagcttattggccgagagatgaaaaatgatagaataaaagtatccctctccatgaaggttgtcaatcaaggggactgggaaagaccttgatcccaacaatgttatcattgagcaagaagagangcggaggcgatccttccaggattacactgggcagnaagatcacccttgaggcttgtcttgaccctacctcaanaagngnggntgtaaagggccctttgcaaaaaatggttatgcancngggggaattaaactttttttccnttgggaaaggaaaggaaagccaatccccantttgnaaacctncctcaggaatcttttaaanaaagagggaaaaaaaanaaccn

SEQ ID NO: 29 - DNA, 408 bp, Homo Sapiens
ctgtcatggctgctcctgtacgtagtcacggtcttgtgctctaaggaaaacgacagcacgtgttctttttcactagtagaagtgacgttggtttcatgttgacaactttgaagccatttggaagtgtttcagtggagaacaaaatgaataacaaagcgggctcctttttctggaaccttagacaattcagtacattagtttcaacaagcagaactatgaggctatgttgtttgggactttgcaaaccaaaaatagttcattcaaactggaacattttaaataactttcataacagaatgcaatcaactgatatcattagatatctctttcaggatgcattcatttttaaatcagatgttggctttcaaacaaagggcataagcctctacagcccttagaattgaagac

SEQ ID NO: 30 - DNA, 414 bp, Homo Sapiens
gtatggcggcgtcaaaggtgaagcaggacatgcctccgccggggggctatgggcccatcgactacaaacggaacttgccgcgtcgaggactgtcgggctacagcatgctggccatagggattggaaccctgatctacgggcactggagcataatgaagtggaaccgtgagcgcaggcgcctacaaatcgaggacttcgaggctcgcatcgcgctgttgccactgttacaggcagaaaccgaccggaggaccttgcagatgcttcgggagaacctggaggaggaggccatcatcatgaaggacgtgcccgactggaaggtgggggagtctgtgttccacacaacccgctgggtgccccccttgatcggggagctgtacggcttgcgcaccacagaggaggctcttcatgccagcc

SEQ ID NO: 31 - DNA, 406 bp, Homo Sapiens
gcagggctttactgcagagcgcgccgggcactccagcgaccgtggggatcagcgtaggtgagctgtggccttttgcgaggtgctgcagccatagctacgtgcgttcgctacgaggattgagcgtctccacccatcttctgtgcttcaccatctacataatgaatcccagtatgaagcagaacaagaagaaatcaaagagaatataaagaatagttctgtcccaagaagaactctgaagatgattcagccttctgcatctggatctcttgttggaagagaaaatgagctgtccgcaggcttgtccaaaaggaaacatcggaatgaccacttaacatctacaacttccagccctggggttattgtcccagaatctagtgaaaataaaatcttggaggagtacccagga

SEQ ID NO: 32 - DNA, 391 bp, Homo Sapiens
gcgaactcggtgaaaggaattggcgccgttcgacaccaggcggatccgctctgcagcacgaacccatctccagccgcagccgcagccgccgcccgggccgaggagcagccgcagcagccgcaccagtggccgagtgagcggagccgagtttgaggcagcgcctagcggtgaatcggggccctcaccatgagttcctcgcctgttaatgtaaaaaagctgaaggtgtcggagctgaaagaggagctcaagaagcgacgcctttctgacaagggtctcaaggccgagctcatggagcgactccaggctgcgctggacgacgaggaggccgggggccgccccgccatggagcccgggaacggcagcctagacctgggcggggattccgctggga

SEQ ID NO: 33 - DNA, 364 bp, Homo Sapiens
  misc_feature (1)...(364): n = g, a, t, or c
cctacggaggtggcagccatctccttctcggcatcatggccgccctcagaccccttgtgaagcccaagatcgtcaaaaagagaaccaagaagttcatccggcaccagtcagaccgatatgtcaaaattaagcgtaactggcggaaacccagaggcattgacaacagggttcgtagaagattcaagggccagatcttgatgcccaacattggttatggaagcaacaaaaaaacaaagcacatgctgcccagtggcttccggaagttcctggtccacaacgtcaaggagctggaaagtgctgctgatgtgcaacaaatcttactgtgccgagatcgctnacaatgtttcttcaagaccgcaaagcc

SEQ ID NO: 34 - DNA, 353 bp, Homo Sapiens
gtcgggtacgcgcacacgttgcatcttcttcctttcgcggggtcctccgtagttctggcacgagccaggcgtactgacaggtggaccagcggactggtggagatggcgacgctctctctgaccgtgaattcaggagaccctccgctaggagctttgctggcagtagaacacgtgaaagacgatgtcagcatttccgttgaagaagggaaagagaatattcttcatgtttctgaaaatgtgatattcacagatgtgaattctatacttcgctacttggctagagttgcaactacagctgggttatatggctctaatctgatggaacatactgagattgatcactggttggagtc

SEQ ID NO: 35 - DNA, 632 bp, Homo Sapiens
  misc_feature (1)...(632): n = g, a, t, or c
gcggagttagcagggctttactgcagagcgcgccgggcactccagcgaccgtggggatcagcgtaggtgagctgtggccttttgcgaggtgctgcagccatagctacgtgcgttcgctacgaggattgagcgtctccacccatcttctgtgcttcaccatctacataatgaatcccagtatgaagcagaaacaagaagaaatcaaagagaatataaagactagttctgtcccaagaagaactctgaagatgattcagccttctgcatctggatctcttgttggaagagaaaatgagctgtccgcaggcttgtccaaaaggaaacatcggaatgaccacttaacatctacaacttccagcctgggggttattgtcccagaattctagtgaaaataaaaatttngnngggagtcacccanggagtatttttgatcttatgattaaaggaaaatccatcttttaatattgaaggggaagngggcagaaaaacggaaaaggggncctttntgaagcacttaagggaaaatgagnaaacttcataaagnaaattgaccaaanggacaattgaaaatggcccgctgaaaaaggaaaataaagactggcnnnaagtagcaaaacatgtccnggtttttg

SEQ ID NO: 36 - DNA, 406 bp, Homo Sapiens
caggcatgttgttgggactggcggccatggagctgaaggtgtgggtggatggcatccagcgtgtggtctgtggggtctcagagcagaccacctgccaggaagtggtcatcgcactagcccaagcaataggccagactggccgctttgtgcttgtgcagcggcttcgggagaaggagcggcagttgctgccacaagagtgtccagtgggcgcccaggccacctgcggacagtttgccagcgatgtccagtttgtcctgaggcgcacagggcccagcctagctgggaggccctcctcagacagctgtccacccccggaacgctgcctaattcgtgccagcctccctgtaaagccacgggcttgcgcttgggctgtgagccccgcaaaacactgaccccgagccagccc

SEQ ID NO: 37 - DNA, 408 bp, Homo Sapiens
  misc_feature (1)...(408): n = g, a, t, or c
ccgagttggaggcggtggagccagcagtaggagtgtgtagagtgcgggattgggggccaggccctgcggagggcgggggaagttgtcttcttttttttccggaggggccggtaaacctggtggctgaacggcaagatgaatttcctccgcggggtaatggggggtcagagtgccggaccccagcacacagaagccgagacgattcaaaagctttgtgacagagtagcttcatctactttattggatgatcgaagaaatgctgttcgtgctctcaaatcattatctaagaaataccgcttggaagtgggtatacaagctatggaacatcttattcatgttttacaaacagatcgttcanattctgaaattataggtatgctttggacacactatataatnnatatctaa

SEQ ID NO: 38 - DNA, 353 bp, Homo Sapiens
acatgctggacccgggtctggatcccgctgcctcggccaccgctgctgccgccgccagccacgacaagggacccgaggcggaggagggcgtcgagctgcaggaaggcggggacggcccaggagcggaggagcagacagcggtggccatcaccagcgtccagcaggcggcgttcggcgaccacaacatccagtaccagttccgcacagagacaaatggaggacaggtgacataccgcgtagtccaggtgactgatggtcagctggacggccagggcgacacagctggcgccgtcagcgtcgtgtccaccgctgcttcgcgggggggcaagcaggctgtgaccaggtgggtgtgc

SEQ ID NO: 39 - DNA, 402 bp, Homo Sapiens
  misc_feature (1)...(402): n = g, a, t, or c
gtcggggtggggcgttcccatgccggcggccgcggggcctggcgtgcgggcgcctccgcgccgcccggggagggggcagtgtcctccgagccaggacaggcatgttgttgggactggcggccatggagctgaaggtgtgggtggatggcatccagctgtgtggtcntgtggggtctcagagcagacacctgccaggaagtggtcatcgcactagcccaagcaataggccagactggccgctttgtgcttgtgcagcggcttcgggagaaggagcggcagttgcttgccacaagagtgtccaagtgggcgcccaggccacctgcggacagtttgccagcgatgtccagtttgtcctgaggcgcacagggcccagcctagctgggaggccttctagacagctgc

SEQ ID NO: 40 - DNA, 397 bp, Homo Sapiens
gagtcgcggcggaaggagcccggccgccgcccgccggcatgagctacgaccgcgccatcaccgtcttctcgcccgacggccacctcttccaagtggagtacgcgcaggaggccgtcaagaagggctcgaccgcggttggtgttcgaggaagagacattgttgttcttggtgtggagaagaagtcagtggccaaactgcaggatgaaagaacagtgcggaagatctgtgctttggatgacaacgtctgcatggcctttgcaggcctcaccgccgatgcaaggatagtcatcaacagggcccgggtggagtgccagagccaccggctgactgtggaggacccggtcactgtggagtacatacccgctacatcgccagtctgaagcagcgttatacgcac

SEQ ID NO: 41 - DNA, 413 bp, Homo Sapiens
gccgaggccagccagtggcacccggaagaaagagacgcggcggcggcgacgccgacaccctcaggacgagtgtccggacttgcccacagcctcaaggaggagacggcgaggcccggcccccgctgtccctggtgtaaagaagtcgccgtagccgtcgcggccgggactccccgggctctcgcccttcaggtttcgttgacactcaggaccgtacgtacgcttgcgccatgttcaagaaactgaagcaaaagatcaagcgaggagcagcagcagctccagcaggcgcttggctcctgctcaggcgtcctccaattcttcaacaccaacaagaatgaggagcaggacatcttcatttcagagcaacttgatgaaggtacacccaatagagagtcaggtgacacacagtcttttga

SEQ ID NO: 42 - DNA, 107 bp, Homo Sapiens
cacccccatcccctctgacagcactcgcaggaaggggggtcgccgtggtcgccgtctgtgaacaagattcctcaaaatattttctgttaataaattgccttcatgta

SEQ ID NO: 43 - DNA, 396 bp, Homo Sapiens
  misc_feature (1)...(396): n = g, a, t, or c
gccgctcaggcgcctgcggctgggtgagcgcacgcgaggcggcgaggcggcagcgtgtttctaggtcgtggcgtcgggcttccggagctttgccggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgatcaagcagaaagtcaagaagacagcggaagctggaggagtncagg

SEQ ID NO: 44 - DNA, 377 bp, Homo Sapiens
taaaggaaagtatgaaatctgcaagctccttttaaaacatggagcagatccaactaaaaagaacagagatggaaatacacctttggatttggtaaaggaaggagacacagatattcaggacttactgagaggggatgctgctttgttggatgctgccaagaagggctgcctggcaagagtgcagaagctctgtaccccagagaatatcaactgcagagacacccagggcagaaattcaacccctctgcacctggcagcaggctataataacctggaagtagctgaatatcttctagagcatggagctgatgttaatgcccaggacaagggtggtttaattcctcttcataatgcggcatcttatgggcatgttgaca

SEQ ID NO: 45 - DNA, 148 bp, Homo Sapiens
gaagaaacaaaagaaagcatcatgatgaataaaatgtctttgcttgtaatttttaaattcatatcaatcatggatgagtctcgatgtgtaggcctttccattccatttattcacactgagtgtcctacaataaacttccgtattttta

SEQ ID NO: 46 - DNA, 413 bp, Homo Sapiens
  misc_feature (1)...(413): n = g, a, t, or c
gcgatgnctattactgcactggggacgtcactgcctggaccaagtgtatggtcaagacacagacacccaaccggaaggagtgggtaaccccaaaggaattccgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcataagcaccaaaaaggaggtggaaaagatgaataagaagatg

SEQ ID NO: 47 - DNA, 286 bp, Homo Sapiens
  misc_feature (1)...(286): n = g, a, t, or c
agaacanggagcatgtgattgaggccctgcgcagggccaagttcaagtttcctggccgccagaagatccacatctcaaaaagtggggcttcaacaagttcaatgctgatgaatttgaagacatggtggctgaaaagcggctcatcccagatggctgtggggtcaagtacatccccagtcgtggccctctggacaagtggcgggccctgcactcatgagggcttccaatgtgctgcccccctcttaatactcaccaataaattctacttcctgtccaaaaaaaaaaa

SEQ ID NO: 48 - DNA, 406 bp, Homo Sapiens
  misc_feature (1)...(406): n = g, a, t, or c
gtcgtggacctcctgcacaagaacatgaaacacctgtggttcttcctcctcctggtggcagctcccagatgggtcctgtcccaggtgcagttacagcagtggggcgcaggactcttgaagccttcggagaccctgtccctcacctgcgcntgtctatggtgggtccttaagtggttatggctggagcntggatccgccagcccccagggaaggggcttggagtggattggggaagtcgaccatcgtggcagcgccaattaccagtcggccctccagagtcgagtctccgtatcattggacacgtccaagaaccaggtctccctgaggctgaactcagtgaccgccgcggacacggctgtttatnctgtgcgagaggcctaatataaagcaatggctctatttgggc

SEQ ID NO: 49 - DNA, 398 bp, Homo Sapiens
gccagatcacgtggagccggggcgccgacaagatcgagggggccattgacattcgtgaaattaaggagatccgcccagggaagacctcacgggactttgatcgctatcaagaggacccagctttccggccggaccagtcacattgctttgtcattctctatggaatggaatttcgcctgaaaacgctgagcctgcaagccacatctgaggatgaagtgaacatgtggatcaagggcttaacttggctgatggaggatacattgcaggcacccacacccctgcagattgagaggtggctccggaagcagttttactcagtggatcggaatcgtgaggatcgtatatcagccaaggacctgaagaacatgctgtcccaggtcaactaccgggtcccaacc

SEQ ID NO: 50 - DNA, 344 bp, Homo Sapiens
gtaccttaccaatgagggtatccagtatctccgtgattaccttcatctgcccccggagattgtgcctgccaccctacgccgtagccgtccagagactggcaggcctcggcctaaaggtctggagggtgagcgacctgcgagactcacaagaggggaagctgacagagatacctacagacggagtgctgtgccacctggtgccgacaagaaagccgaggctggggctgggtcagcaaccgaattccagtttagaggcggatttggtcgtggacgtggtcagccacctcagtaaaattggagaggattcttttgcattgaataaacttacagccaaaaaaacctta

SEQ ID NO: 51 - DNA, 415 bp, Homo Sapiens
aatccgggcaccaggttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattcctgagaaatctcttacctcaagaaattgaaggttaaaaaacaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggcatgattgagaaactcggggggaagttgacgggga

SEQ ID NO: 52 - DNA, 412 bp, Homo Sapiens
aggctatgtgttttgtcagggggttgagaatgagtgtgaggcgtattataccatagccgcctagtttcaagagtactgcggcaagtactattgacccagcgatgggggcttcgacatgggctttagggagtcataagtggagtccgtaaagaggtatctttactataaaggctattgtgtaagctagtcatattaagttgttggctcaggagtttgatagttcttgggcagtgagagtgagtagtagaatgtttagtgagcctagggtgttgtgagtgtaaattaagtgcgatgagtaggggaagggagcctactagggtgtagaataggaagtatgtcctgcgttcaggcgttctgctggttgcctcatcgggtgatgatagccaaggtggggataagtgtggttccaaac

SEQ ID NO: 53 - DNA, 394 bp, Homo Sapiens
gagcgatgggcatctctcgggacaactggcacaagcgccgcaaaaccgggggcaagagaaagccctaccacaagaagcggaagtatgagttggggcgcccagctgccaacaccaagattggcccccgccgcatccacacagtccgtgtgcggggaggtaacaagaaataccgtgccctgaggttggacgtggggaatttctcctggggctcagagtgttgtactcgtaaaacaaggatcatcgatgttgtctacaatgcatctaataacgagctggttcgtaccaagaccctggtgaagaattgcatcgtgctcatcgacagcacaccgtaccgacagtggtacgagtcccactatgcgctgccctgggccgcaagaagggagccaagctgact

SEQ ID NO: 54 - DNA, 609 bp, Homo Sapiens
  misc_feature (1)...(609): n = g, a, t, or c
ggcacattggctaaaggctctttggagaatgttttggattccaaagacaaaacccaaaagagcaatggtgaaaagaatgaaaaatgtgagaccaaagagaaaggagcaatcacagcaaaggaactatacacaatgatgacggataaaaacatcagcttgattataatggatgctcgaagaatgcaggattatcaggattcctgtattttacattctctcagtgttcctgaagaagccatcagtccaggagtcactgctagttggattgaagcacacctgccagatgattctaaagacacatggaagaagagggggnaatgtggagtattgtgggtacttcttgactgggtttaagttctgccaaagatttaccagattggaaccaactctcccggagtttgaaagatgcacttttcaggggggaaagtaaaactggtcctgcncatgagcctttggntttaanggggggtttgaaactggtcctttttntnccccgtttccacaagcttangggcntcccccncacccnanaaaanngggnttcatngggtttnctttcccttnggaaaaaaaatcttttaaacggggnccaccccccctttttaaaan

SEQ ID NO: 55 - DNA, 694 bp, Homo Sapiens
  misc_feature (1)...(694): n = g, a, t, or c
taccntgtttgngctcgcgcgcctgcaggtcgacactagtggatccaaagcaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttaagagtgcccgccttagacggagcanggagttttcgttagaaaagcggacgctgttctaaaaaangtctctggcagatctgtctgggctggtgatgacnaatattatgaaaatgtgncctttntgaanaaaatggggttagcttcnaactttctttcgcaagggttcaagtttttatttnccttgggaatncctggggaacccccggggaaggggggatgccngancaaaggnttttgtttagccnnaaggggaccttgcggactncacggggaaatttntttgttt

SEQ ID NO: 56 - DNA, 180 bp, Homo Sapiens
gtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaa

SEQ ID NO: 57 - DNA, 645 bp, Homo Sapiens
  misc_feature (1)...(645): n = g, a, t, or c
gggacatctccagcaccctcatcgccctggccgacaagcacgcaaccctgtcagtctgctataaggccggaccgggggcggacaacggcgaggaaggggaaatagaagaggaaatggagaatccggaaatggtggacctgcccgagaaactaaagcaccagctgcggcatcgggagctgttcctctcccggcagctggagtctctgcccgccacgcacatcaggggcaagtgcagcgtcaccctgctcaacgagaccgagtcgctcaagtcctacctggagcgggaggatttcttcttctattctctagtctacgacccacagcagaagaccctgctggcagataaaggagagattcgagtaggaaaccggtaccaggcagacatcaccgacttgttaaaagaaggcgaggaggatggccgagaccagtccaggtttggagacccaagtgtngggaggggcacaacccacttacagacaagccagatgnncattcttgggnggngggccgcttttggggcaccttccacgggncctggactgaganntttcttccacacccacttgcaaagancnccnaattgcttccnaaaatannccttttccncccctgggttctttcaaaanaaatttacaaatttcaggc

SEQ ID NO: 58 - DNA, 650 bp, Homo Sapiens
  misc_feature (1)...(650): n = g, a, t, or c
ccgaggccagccagtggcacccggaagaaagagacgcggcggcggcgacgccgacaccctcaggacgagtgtccggacttgcccacagcctcaaggaggagacggcgaggcccggcccccgctgtccctggtgtaaagaagtcgccgtagccgtcgcggccgggactccccgggctctcgcccttcaggtttcgttgacactcaggaccgtacgtacgctgcgccatgttcaagaaactgaagcaaaagatcagcgaggagcagcagcagctccagcaggcgctggctcctgctaggcgtcctccaattcttcaacaccaacaagaatgaggagcaggacatcttcatttacagagcaacttgatgnaaggtacacccaatagaagagttcaaggtggacacacaagtcttttgcacagaaagcttcagttccnggtgccctcgggggagtctttgttttngaagtccgataaggaattnttttccggncttttttaaagagttttttggtccaaaatntttcaaaaaatcctgaatgattgactggaagntctgccgttttgatcccccttttttgatgnngggtaaaaattgggggggatttnanacgnttaaaaaaaaatgttttcnggttgnaaaaaagaanaann

SEQ ID NO: 59 - DNA, 615 bp, Homo Sapiens
  misc_feature (1)...(615): n = g, a, t, or c
agaaaacacaaaagaaattccggaagcgagaagagaaggctgctgagcacaaggccaagtccttgggggagaaatctccagcagcctctggggccaggaggcctgaggcagccaaagaggaagcagcttgggcttccagctcagcagggaaccctgcagatggcctggccactgagcctgagtctgtctttgctctggatgttctgcgacagcgactgcatgagaagatccaggaggcccggggccagggcagtgccaaggagctgtcccctgccgccttggagaaaaggcggcggagaaagcaggaacgggaccggaagaagaggaagcgaaaggagctgcgggcgaaaganaagccaggaaggctgaggaggccacggaggcccaggaggtggtggaggcaaccccagagggggcctgcacggaccncangagcccccggcttgtcttcattaggggggaggtgagcgaaacaaccggccacaaggggcaccaaaaaaaaaaaagcanaggtgaagggaacctcnccctnccggagaataccgcanttttgaacctgcgcnccgaaaaccgttganaantcgcccnatgggggaagcccngactgggcaaana

SEQ ID NO: 60 - DNA, 640 bp, Homo Sapiens
  misc_feature (1)...(640): n = g, a, t, or c
ggtctctcgtcgctgcaggcgcctcagcccagccgcgtgccttggcccatggccgcctactcttaccgccccggccctggggccggccctgggcctgctgcaggcgcggcgctgccggaccagagcttcctgtggaacgttttccagagggtcgataaagacaggagtggagtgatatcagacaccgagcttcagcaagctctctccaacggcacgtggactccctttaatccagtgactgtcaggtcgatcatatccatgtttgaccgtgagaacaaggccggcgtgaacttcagcgagttcacgggtgtgtggaagtacatcacggactggcagaacgtcttccgcacgtacgaccgggacaactccgggatgatcgataagaacgagctgaagcaggccctctcaggctaccggcttntntgaccagttccacgacatcctattcgaaaagtttgacaggcagggacggggcaaaatcgcttcaacactttatcanggctgnattgtctgaanaggtggcggtnttttaaacttcacccggataggangtgtttaaggggtcacnaaaaanctgccangntttaaaantcgaagacngccccttgggagggccccactnggaaggccaatgtnccnt

SEQ ID NO: 61 - DNA, 628 bp, Homo Sapiens
  misc_feature (1)...(628): n = g, a, t, or c
gcgcagggatggcacaaaagaaatatcttcaagcaaaattgacccagtttttaagggaagacaggattcaactttggaaacctccatatacagatgaaaataaaaaagttggtttggcattaaaggaccttgctaagcagtactctgacagactagaatgctgtgaaaatgaagtagaaaaggtaatagaagaaatacgttgcaaggcaattgagcgtggaacaggaaatgacaattatagaacaacgggaattgctacaatcgaggtgtttttaccaccaagactaaaaaaagataggaaaaacttgttggagacccgattgcacatcactggcagagaactgaggtccaaaatagctgaaacctttggacttcaagaaaattatatcaaaattgtcataaataagaagcaactacactagggaaaacccttgaagaaaaggcgtggctccaatgtgaaagcgatggtgcttgactaaaacatctgaaaggacgcaggaaacttccgttggggaagagagcaaaanaggccactcaagaaaacantcgngncaganggcttgaatctggcagaagcacnaaagngggggaccaaagaacccnctttaacttnttacngncggcnatn

SEQ ID NO: 62 - DNA, 614 bp, Homo Sapiens
  misc_feature (1)...(614): n = g, a, t, or c
ggctctggcacacagctgtgctcacaaaatactgggtggcttggttagagctaattgtagtggagcctgcaggtgagggtgagggagggggctgcaggtcaggtaagatctggaagacagacgtcagcttggagggcagggggactctaaggcaaggagatttacagttgggaaggaggcagtggcagaggggtgagggacaggggcccttaagtccagcgaggaaagctcggtgtgggcccgctctacgctccgtttggggtgacctggaacgcctcttctcccagctccctccagccatcagcagcctcttgtcaagcttctgcctcgccccagtctatccccaaccccaaatcaagaccacctttcttcacggtcactatttattctttggtccttttctttttgtaagaaacattcacaaaaaccagtgccnnncccnnnnnnnnnnnnnnnnnnnnnnnnnnnnnnnnnnnnnnnnnnnnaaaaactcgggagtcttttaagggggcgnggccntngntttccccgggggggcccggnaaaggnccccatncctttngggggggggttnnatntgggcccggnttaaaacntngatngnaccnctggct

SEQ ID NO: 63 - DNA, 618 bp, Homo Sapiens
  misc_feature (1)...(618): n = g, a, t, or c
gcagccagggtcggtgaaggatcccaaaatggctgggcgaaaacttgctctaaaaaccattgactgggtagcttttgcagagatcataccccagaaccaaaaggccattgctagttccctgaaatcctggaatgagaccctcacctccaggttggctgctttacctgagaatccaccagctatcgactgggcttactacaaggccaatgtggccaaggctggcttggtggatgactttgagaagaagtttaatgcgctgaaggttcccgtgccagaggataaatatactgcccaggtggatgccgaagaaaaagaagatgtgaaatcttgtgctgagtggggtgtctctctcaaaggccaggattgtagaatatgaagaaagagatggagaagatgaaagaacttaattncttttgatcagatgaccattganggacttgaatgaagcttttccagaaaccaaattagacaagaaaaagtntcctattggnctcaccanccattgggaattataaaatgagtcnggaggaagtttggccttgntaccatttggccttaaatattattttcccnnnnnnnnnnnnnnnnnnnnnnnnnnnnnaaaaacctcggggnctt

SEQ ID NO: 64 - DNA, 244 bp, Homo Sapiens
gctgtcaagcagttccacgactccaagatcaagttcccgctgccccaccgggtcctgcgccgtcagcacaagccacgcttcaccaccaagaggcccaacaccttcttctaggtgcagggccctcgtccgggtgtgccccaaataaactcaggaacgccccaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 65 - DNA, 407 bp, Homo Sapiens
gttgctgcttctcagatgccaagactatgtatgaggttttccaaagaggactcgctgtgtctgacaatgggccctgcttgggatatagaaaaccaaaccagccctacagatggctatcttacaaacaggtgtctgatagagcagagtacctgggttcctgtctcttgcataaaggttataaatcatcaccagaccagtttgtcggcatctttgctcagaataggccagagtggatcatctccgaattggcttgttacaccgtactctatggtagcttgtacctctgtatgacaccttgggaccagaagccatcgtacatattgtcaacaaggctgatatcgccgtggtgatctgtgacacaccccaaaaggcattggtgctgatagggaatgtaagaaggctcaccc

SEQ ID NO: 66 - DNA, 402 bp, Homo Sapiens
  misc_feature (1)...(402): n = g, a, t, or c
ccggatngggtctccaggctggcgagcgcccaggccagactggccgctttgtgcttgtgcagcggcttcgggagaaggagcggcagttgctgccacaagagtgtccagtgggcgcccaggccaccctgcggacagtttgccagcgatgtccagtttgtcctgaggcgcacagggcccagcctagctgggaggccctcctcagacagctgtccacccccggaacgctgcctaattcgtgccagcctccctgtaaagccacgggctgcgctgggctgtgagccccgcaaaacactgacccccgagccagcccccagcctctcacgccctgggcctgcggcccctgtgacacccacaccaggctgctgcacagacctgcgggcctgaactcagggtgcagaggac

(19) United States
(12) Patent Application Publication
(10) Pub. No.: US 2003/0180738 A1
(43) Pub. Date: Sep. 25, 2003

(54) Cancer associated genes and their products

(76) Inventors: Robert Charles Rees, Sheffield (GB); Geng Li, Nottingham (GB); Shahid Mian, Nottingham (GB)

Correspondence Address: BANNER & WITCOFF, 1001 G STREET N W, SUITE 1100, WASHINGTON, DC, 20001, US

(21) Appl. No.: 10/181,447

(22) PCT Filed: Jan. 18, 2001
(86) PCT No.: PCT/GB01/00188

(30) Foreign Application Priority Data
Jan. 18, 2000 (GB) 0000993.6

(51) Int. Cl. C12Q 1/68; C07H 21/04

(52) U.S. Cl. 435/6; 536/24.3

(57) ABSTRACT

The application discloses cancer-associated genes and their products, especially those identifiable by SEREX. The genes and products are used to identify, track and treat cancer. Preferably the cancer is prostate cancer.

The invention relates to isolated nucleic acid sequences which are expressed in cancers, especially prostate cancers, to their protein products and to the use of the nucleic acid and protein products for the identification and treatment of prostate cancers.

The prostate gland is an accessory sex gland in males which is wrapped around the urethra as this tube leaves the bladder. The gland secretes an alkaline fluid during ejaculation. Cancer of the prostate gland is very serious and represents the second leading cause of death from cancer in men.

Two specific proteins are known to be made in very high concentrations in prostate cancer cells. These are prostatic acid phosphatase (PAP) and prostate specific antigen (PSA). These proteins have been characterised and have been used to follow response to therapy. However, it has been difficult to correlate the presence of these two proteins to the presence of cancer.

Accordingly, there is a need to identify new genes and proteins which are associated with the presence of prostate cancer.

The inventors have used a technique known as SEREX (Serological Identification of Antigens by Recombinant Expression Cloning) to identify genes which are over-expressed in prostate cancer tissue. This technique was published by Sahin et al (PNAS (USA), 1995, Vol. 92, pages 11810-11813). SEREX uses total RNA isolated from tumour biopsies from which poly(A)⁺ RNA is then isolated. cDNA is then produced using an oligo (dT) primer. The cDNA fragments produced are then cloned into a suitable expression vector, such as a bacteriophage and cloned into a suitable host, such as E. coli. The clones produced are screened with high-titer IgG antibodies in autologous patient serum, to identify antigens associated with the tumour.

The inventors have used this technique to identify a number of genes and gene products associated with prostate cancer. Furthermore, preliminary results have found that some antigens identified by this technique have been also identified by the inventors as being associated with other cancers, such as stomach cancer and oesophagial cancer.

A first aspect of the invention provides an isolated mammalian nucleic acid molecule selected from SEQ.ID. 1, SEQ.ID. 2, SEQ.ID. 3, SEQ.ID. 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66. Preferably the isolated nucleic acid molecule encodes a mammalian antigen which is expressed in higher than normal concentrations in cancer cells, compared with normal non-cancerous cells. Preferably the cancer is prostate cancer. The term “higher than normal concentrations” preferably means that the protein is expressed at a concentration at least 5 times greater in tumour cells than normal cells.

The invention also includes, within its scope, nucleic acid molecules complementary to such isolated mammalian nucleic acid molecules.

The nucleic acid molecules of the invention may be DNA, cDNA or RNA. In RNA molecules “T” (Thymine) residues may be replaced by “U” (Uridine) residues.

Preferably, the isolated mammalian nucleic acid molecule is an isolated human nucleic acid molecule.

The invention further provides nucleic acid molecules comprising at least 15 nucleotides capable of specifically hybridising to a sequence included within the sequence of a nucleic acid molecule according to the first aspect of the invention. The hybridising nucleic acid molecule may either be DNA or RNA. Preferably the molecule is at least 90% homologous to the nucleic acid molecule according to the first aspect of the invention. This may be determined by techniques known in the art.

The term “specifically hybridising” is intended to mean that the nucleic acid molecule can hybridise to nucleic acid molecules according to the invention under conditions of high stringency. Typical conditions for high stringency include 0.1× SET, 0.1% SDS at 68° C. for 20 minutes.

The invention also encompasses variant DNAs and cDNAs which differ from the sequences identified above, but encode the same amino acid sequences as the isolated mammalian nucleic acid molecules, by virtue of redundancy in the genetic code.

| | U | C | A | G | |
|---|---|---|---|---|---|
| U | 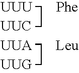 | 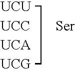 | 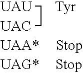 | 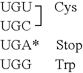 | 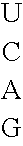 |
| C | 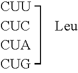 | 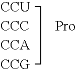 | 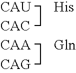 | 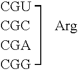 | 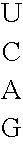 |
| A | 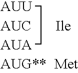 | 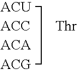 | 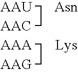 | 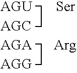 | 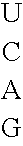 |
| G | 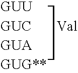 | 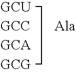 | 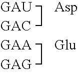 | 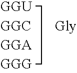 | 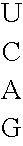 |

*Chain-terminating, or “nonsense” codons.
**Also used to specify the initiator formyl-Met-tRNAMet. The Val triplet GUG is therefore “ambiguous” in that it codes both valine and methionine.

The genetic code showing mRNA triplets and the amino acids which they code for.

The invention also includes within its scope vectors comprising a nucleic acid according to the invention. Such vectors include bacteriophages, phagemids, cosmids and plasmids. Preferably the vectors comprise suitable regulatory sequences, such as promoters and termination sequences which enable the nucleic acid to be expressed upon insertion into a suitable host. Accordingly, the invention also includes hosts comprising such a vector. Preferably the host is E. coli.

A second aspect of the invention provides an isolated protein or peptide obtainable from a nucleic acid sequence according to the invention. As indicated above, the genetic code for translating a nucleic acid sequence into an amino acid sequence is well known.

The invention further provides polypeptide analogues, fragments or derivatives of antigenic polypeptides which differ from naturally-occurring forms in terms of the identity of location of one or more amino acid residues (deletion analogues containing less than all of the residues specified for the protein, substitution analogues wherein one or more residues specified are replaced by other residues in addition analogues wherein one or more amino acid residues are added to a terminal or medial portion of the polypeptides) and which share some or all properties of the naturally-occurring forms. Preferably such polypeptides comprise between 1 and 20, preferably 1 and 10 amino acid deletions or substitutions.

Preferably the protein or peptide is at least 95%, 96%, 97%, 98% or 99% identical to the sequences of the invention. This can be determined conventionally using known computer programs such as the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The nucleic acids and proteins/peptides of the invention are preferably identifiable using the SEREX method. However, alternative methods, known in the art, may be used to identify nucleic acids and protein/peptides of the invention. These include differential display PCR (DD-PCR), representational difference analysis (RDA) and suppression subtracted hybridisation (SSH).

All of the nucleic acid molecules according to the invention and the peptides which they encode are detectable by SEREX (discussed below). The technique uses serum antibodies from prostate cancer patients to identify the molecules. It is therefore the case that the gene products identified by SEREX are able to evoke an immune response in a patient and may be considered as antigens suitable for potentiating further immune reactivity if used as a vaccine.

The third aspect of the invention provides the use of nucleic acids or protein/peptides according to the invention, to detect or monitor prostate cancer.

The use of a nucleic acid molecule hybridisable under high stringency conditions, a nucleic acid according to the first aspect of the invention to detect or monitor prostate cancer is also encompassed. Such molecules may be used as probes, e.g. using PCR.

The expression of genes, and detection of their protein products and/or peptides may be used to monitor disease progression during therapy or as a prognostic indicator of the initial disease status of the patient. There are a number of techniques which may be used to detect the presence of a gene, including the use of Northern blot and reverse transcription polymerase chain reaction (RT-PCR) which may be used on tissue or whole blood samples to detect the presence of cancer associated genes. For protein and/or peptide sequences in-situ staining techniques or enzyme linked ELISA assays or radio-immune assays may be used. RT-PCR based techniques would result in the amplification of messenger RNA of the gene of interest (Sambrook, Fritsch and Maniatis, Molecular Cloning, A Laboratory Manual, 2^{nd} Edition). ELISA based assays necessitate the use of antibodies raised against the protein or peptide sequence and may be used for the detection of antigen in tissue or serum samples (McIntyre C. A., Rees R. C. et. al., Europ. J. Cancer 28, 58-631 (1990)). In-situ detection of antigen in tissue sections also rely on the use of antibodies, for example, immuno peroxidase staining or alkaline phosphatase staining (Gaepel, J. R., Rees, R. C. et.al., Brit. J. Cancer 64, 880-883 (1991)) to demonstrate expression. Similarly radio-immune assays may be developed whereby antibody conjugated to a radioactive isotope such as I¹²⁵ is used to detect antigen in the blood (Turkes, A., et. al., Prostate-specific antigen—problems in analysis. Europ. J. Cancer. 27, 650-652 (1991)).

Blood or tissue samples may be assayed for eleviated concentrations of the nucleic acid molecules, proteins or peptides.

Kits for detecting or monitoring cancer, such as prostate cancer, using polypeptides, nucleic acids or antibodies according to the invention are also provided. Such kits may additionally contain instructions and reagents to carry out the detection or monitoring.

The fourth aspect of the invention provides for the use of nucleic acid molecules according to the first aspect of the invention or protein/peptide molecules according to the second aspect of the invention in the prophylaxis or treatment of cancer, or pharmaceutically effective fragments thereof. By pharmaceutically effective fragment, we mean a fragment of the molecule which still retains the ability to be a prophylactant or to treat cancer. The cancer may be prostate cancer.

The molecules are preferably administered in a pharmaceutically amount. Preferably the dose is between 1 μg/kg. to 10 mg/kg.

The nucleic acid molecules may be used to form DNA-based vaccines. From the published literature it is apparent that the development of protein, peptide and DNA based vaccines can promote anti-tumour immune responses. In pre-clinical studies, such vaccines effectively induce a delayed type hypersensitivity response (DTH), cytotoxic T-lymphocyte activity (CTL) effective in causing the destruction (death by lysis or apoptosis) of the cancer cell and the induction of protective or therapeutic immunity. In clinical trials peptide-based vaccines have been shown to promote these immune responses in patients and in some instances cause the regression of secondary malignant disease. Antigens expressed in prostate cancer (or other types of cancers) but not in normal tissue (or only weakly expressed in normal tissue compared to cancer tissue) will allow us to assess their efficacy in the treatment of cancer by immunotherapy. Protein or peptide derived from the tumour antigen may be administered with or without immunological adjuvant to promote T-cell responses and induce prophylactic and therapeutic immunity. DNA-based vaccines preferably consist of part or all of the genetic sequence of the tumour antigen inserted into an appropriate expression vector which when injected (for example via the intramuscular, subcutaneous or intradermal route) cause the production of protein and subsequently activate the immune system. An alternative approach to therapy is to use antigen presenting cells (for example, dendritic cells, DC's) either mixed with or pulsed with protein or peptides from the tumour antigen, or transfect DC's with the expression plasmid (preferably inserted into a viral vector which would infect cells and deliver the gene into the cell) allowing the expression of protein and the presentation of appropriate peptide sequences to T-lymphocytes.

Accordingly, the invention provides a nucleic acid molecule according to the invention in combination with a pharmaceutically-acceptable carrier.

A further aspect of the invention provides a method of prophylaxis or treatment of prostate cancer comprising the administration to a patient of a nucleic acid molecule according to the invention.

The protein/peptide molecules according to the invention may be used to produce vaccines to vaccinate males against prostate cancer.

Accordingly, the invention provides a protein or peptide according to the invention in combination with a pharmaceutically acceptable carrier.

The invention further provides use of a protein or peptide according to the invention in a prophylaxis or treatment of a cancer such as prostate cancer.

Methods of prophylaxis or treating prostate cancer, by administering a protein or peptide according to the invention to a patient, are also provided.

Vaccines comprising nucleic acid and/or proteins and peptides according to the invention are also provided.

The proteins and peptides of the invention may be used to raise antibodies. In order to produce antibodies to tumour-associated antigens procedures may be used to produce polyclonal antiserum (by injecting protein or peptide material into a suitable host) or monoclonal antibodies (raised using hybridoma technology). In addition PHAGE display antibodies may be produced, this offers an alternative procedure to conventional hybridoma methodology. Having raised antibodies which may be of value in detecting tumour antigen in tissues or cells isolated from tissue or blood, their usefulness as therapeutic reagents could be assessed. Antibodies identified for their specific reactivity with tumour antigen may be conjugated either to drugs or to radioisotopes. Upon injection it is anticipated that these antibodies localise at the site of tumour and promote the death of tumour cells through the release of drugs or the conversion of pro-drug to an active metabolite. Alternatively a lethal effect may be delivered by the use of antibodies conjugated to radioisotopes. In the detection of secondary/residual disease, antibody tagged with radioisotope could be used, allowing tumour to be localised and monitored during the course of therapy.

The term “antibody” includes intact molecules as well as fragments such as Fa, F(ab′)₂ and Fv.

The invention accordingly provides a method of treating prostate cancer by the use of one or more antibodies raised against a protein or peptide of the invention.

The cancer-associated proteins identified may form targets for therapy.

The invention also provides nucleic acid probes capable of binding sequences of the invention under high stringency conditions. These may have sequences complementary to the sequences of the invention and may be used to detect mutations identified by the inventors. Such probes may be labelled by techniques known in the art, e.g. with radioactive or fluorescent labels.

The invention will now be described by reference to the following figure and examples:

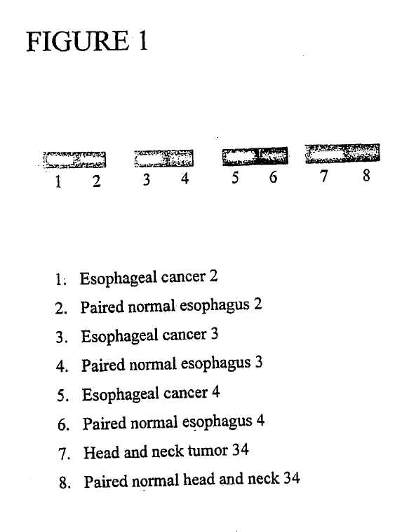

FIG. 1 shows RT-PCR of different tumour samples showing over-expression of MTA-1 (SEQ.ID. 57).

### TECHNIQUE USED TO IDENTIFY GENES ENCODING TUMOUR ANTIGENS (SEREX TECHNIQUE)

The technique for the expression of cDNA libraries from human prostate cancer tissue is described, and was performed according to published methodology (Sahin et.al. Proc Natl. Acad. Sci. 92, 11810-11813, 1995).

SEREX has been used to analyze gene expression in tumour tissues from human melanoma, renal cell cancer, astrocytoma, oesophageal squamous cell carcinoma, colon cancer, lung cancer and Hodgkin's disease. Sequence analysis revealed that several different antigens, including HOM-MEL-40, HOM-HD-397, HOM-RCC-1.14, NY-ESO-1, NY-LU-12, NY-CO-13 and MAGE genes, were expressed in these malignancies, demonstrating that several human tumour types express multiple antigens capable of eliciting an immune response in the autologous host. This represents an alternative and more efficient approach to identify tumour markers, and offers distinct advantages over previously used techniques:

1) the use of fresh tumour specimens to produce the cDNA libraries obviates the need to culture tumour cells in vitro and therefore circumvents artefacts, such as loss or neo-antigen expression and genetic and phenotypic diversity generated by extended culture;

2) the analysis is restricted to antigen-encoding genes expressed by the tumour in vivo;

3) using cDNA expression cloning, the serological analysis (in contrast to autologous typing) is not restricted to cell surface antigens, but covers a more extensive repertoire of cancer-associated proteins (cytosolic, nuclear, membrane, etc.);

4) in contrast to techniques using monoclonal antibodies, SEREX uses poly-specific sera to scrutinise single antigens that are highly enriched in lytic bacterial plaques allowing the efficient molecular identification of antigens following sequencing of the cDNA. Subsequently the tissue-expression spectrum of the antigen can be determined by the analysis of the mRNA expression patterns using northern blotting and reverse transcription-PCR (RT-PCR), on fresh normal and malignant (autologous and allogeneic) tissues. Likewise, the prevalence of antibody in cohorts of cancer patients and normal controls can be determined.

Construction of cDNA Expression Libraries, Screening and Sequencing

The detailed methodology for SEREX expression cloning established by the inventors is as follows: Total RNA is isolated from fresh prostate cancer tissues using the guanidinium thiocyanate-phenol-chloroform extraction method; RNA integrity is determined by electrophoresis in formalin/MOPS gels. Poly(A)+ RNA is prepared by applying the prepared RNA sample to a column of oligo (dT) cellulose and cDNA expression libraries is constructed from 5-8 μg of poly(A)+ RNA; first-strand synthesis is performed using an oligo(dT) primer with an internal Xho I site and 5-methyl-CTP. cDNA is ligated to EcoRI adaptors and digested with Xho I and cDNA fragments are cloned directionally into the bacterophage expression vector, packaged into phage particles, and used to transfect Escherichia coli. Immuno-screening for the detection of clones reactive with antibodies present in diluted autologous serum is then performed. Transfection for primary screening and plaque transfer onto nitrocellulose membranes is followed by pre-incubation of the membranes with an alkaline phosphatase-conjugated antibody specific for human IgG. Reactive clones representing expressed IgG heavy chains visualized by staining are eliminated from the study. These pre-stained membranes are then incubated with the autologous patient serum, and binding to recombinant proteins expressed in lytic plaques detected by incubation with an alkaline phosphatse-conjugated goat anti-human IgG, and differentiated from the IgG-heavy chain transcripts. The reactive clones are sub-cloned, purified, and in vitro excised to pBK-CMV plasmid forms. Plasmid DNA is prepared using the Wizard (Trade Mark) Miniprep DNA purification system (Promega Corp., Southampton, UK). The inserted DNA is evaluated by restriction mapping, and clones representing different cDNA inserts sequenced using the automated sequencer.

Expression of Antigens in Different Cancers

The expression of metastasis associated 1 (MTA1) (SEQ.ID. 57) in cancer samples was compared with that in corresponding normal tissues by semi-quantitative reverse-transcription polymerase chain reaction (RT-PCR). RT-PCR was carried out using processes well known in the literature. A relative over-expression of MTA1 mRNA (normal/tumour ratio≧2) was observed in esophageal cancer (3/7) and head and neck tumour (1/7) (Table 1). See FIG. 1, tracks 6 and 8. Testis did not show any over-expression. GAPDH (Glyceraldehyde-3-phosphate Dehydrogenase) expression was also tested as a control. No difference in expression was normal tissue and observed between tumours.

TABLE 1

| Tumour type | Positive rate |
|---|---|
| Esophageal cancer | 3/7 |
| Head and neck tumour | 1/7 |

Table 2 shows the results of further studies of a variety of sequences in different tumours. “-” indicates not studied. This table shows that the proteins are immunogenic in a higher portion of patients with cancer than controls since the patients have antibodies against the cloned protein product.

TABLE 2
Serological responses in cancer patients and controls to the protein products of genes
cloned from the DNA library using SEREX
Immunoscreening with sera from:

| SEQ ID # | Gene | size bp | Identity | Controls | BPH | Prostate Ca | Head & Neck Ca | Co Ca | Ga Ca |
|---|---|---|---|---|---|---|---|---|---|
| 8 | Pr III-41 | 3500 | Unknown | 0/7 | — | 4/10 | 2/4 | — | — |
| | 137,144 | | | | | | | | |
| 10 | Pr III-90 | 3000 | Unknown | 0/10 | 0/2 | 2/7 | 2/4 | — | — |
| | 102,108 | | | | | | | | |
| 12 | Pr III-104 | 1500 | Unknown | 0/8 | 0/2 | 2/7 | 2/4 | — | — |
| 16 | Pr III-133 | 1550 | Unknown | 0/5 | 0/2 | 4/7 | — | — | — |
| 18 | Pr III-147 | 1100 | Unknown | 1/10 | 0/2 | 8/12 | 2/4 | 0/2 | — |
| 50 | Pr III-157 | 400 | Hu Ribosomal | 0/4 | — | 5/10 | 1/4 | — | — |
| | | | Protein S10 | | | | | | |
| 57 | Pr III-176 | 2600 | MTA1 | 0/13 | 0/3 | 2/13 | — | 0/2 | 0/2 |
| 60 | Pr III-197 | 1200 | ALG2 | 1/17 | 0/3 | 4/13 | 3/4 | 0/2 | 0/2 |
| 29 | Pr III-213 | 2500 | Unknown | 0/6 | 0/2 | 4/12 | 2/4 | 0/2 | — |

Serum samples from:
Controls
BPH—Benign Prostatic Hyperplasia
Prostate Cancer
Head and Neck Cancers
Co Ca—Colon Cancer
Ga Ca—Gastric Cancer

Table 3 shows some of the mutations identified by the inventors.

TABLE 3

| SEQ ID # | Gene | Identity | Mutation |
|---|---|---|---|
| 35 | PrIII-30 | Human geminin. | Point mutation at nt 78 |
| | | | (A to C) |
| 34 | PrIII-13 | Human glutamyl- | 261 nt longer at 5′ of mRNA. |
| | | prolyl-tRNA | There is a starting code |
| | | synthetase | (ATG) in this region. This |
| | | | clone may be a new isoform. |
| 43 | PrIII-118 | Human poly | Point mutation at nt 79 |
| | | (ADP-ribose) | (C to G) and nt 145 (G to A). |
| | | polymerase | |
| | | mRNA. | |
| 44 | PrIII-119 | Human tankyrase | Point mutation at nt 2410 |
| | | | (G to A). |
| 52 | PrIII-163 | Human | Point mutation at nt 10769 |
| | | mitochodrial | (A to G). |
| | | DNA | |
| 60 | PrIII-197 | Human calcium | 6 nt deletion from nt 487 to |
| | | binding protein | 492 (GGTTTC). |
| | | (ALG-2) mRNA. | |
| 65 | PrIII-219 | Human FACL5 | Point mutation at nt 758 |
| | | for fatty acid | (A to G) |
| | | coenzyme A | |
| | | ligase 5 | |
| 66 | PrIII-224 | Human DNA- | 129 nt deletion in exon 2. |
| | | binding protein | This clone may be an |
| | | (HRC 1) mRNA | alternatively spliced isoform. |

Mutations detected in the sequence of genes cloned by SEREX.

| | |
|---|---|
| PR2-7A Human mRNA for KIAA0160 gene | |
| GGCGGCTCGGGGCCCAGCGCGGGGTCCGGGGGAGGCGGCTTCGGGGGTTCGGCGGCGGT | SEQ ID 1 |
| GGCGGCGGCGACGGCTTCGGGCGGCAAATCCGGCGGCGGGAGCTGTGGAGGGGGTGGCA | |
| GTTACTCGGCCTCCTCCTCCTCCTCCGCGGCGGCAGCGGCGGGGGCTGCGGTGTTACCGGT | |
| GAAGAAGCCGAAAATGGAGCACGTCCAGGCTGACCACGAGCTTTTCCTCCAGGCCTTTGA | |
| GAAGCCAACACAGATCTATAGATTTCTTCGAACTCGGAATCTCATAGCACCAATATTTTTG | |
| CACAGAACTCTTACTTACATGTCTCATCGAAACTCCAGAACAAACATCAAAAGGAAAACA | |
| TTTAAAGTTGATGATATGTTATCAAAAGTAGAGAAAATGAAAGGAGAGCAAGAA | |
| PR2-1A Human protein immuno-reactive with anti-PTH polyclonal | |
| antibodies mRNA | |
| ACAGGTGAAAAACCAAATACTTTCTAGGGATGACCTTGATGACATAATTCAGTCATCTCA | SEQ ID 2 |
| AACAGTCTCAGAGGACGGTGACTCGCTTTGCTGTAATTGTAAGAATGTCATATTACTCATT | |
| GATCAACATGAAATGAAGTGTAAAGATTGTGTTCACCTATTGAAAATTAAAAAGCATTTT | |
| GTTTATGTAAAAGATTAACAGAACTTAAAGATAATCACTGTGAGCAACTTAGAGTAAAAA | |
| TTCGAAAACTGAAAAAATAAGGCTAGTGTACTACAAAAGAGACTATCTGAAAAAGAAGAA | |
| ATAAAATCGCAGTTAAAGCATGAAACACTTGAATTGGAAAAAGAACTCTGTAGTTTGAGA | |
| TTTGGCCTACAGCAAGAAAAAAAGAAAAGAAGAAATGTTGA | |
| PR2-21 2 Human JK-recombination signal binding protein (RBPJK) | |
| gene | |
| GAGAGTTTGTGGAAGATGGCGCCTGTTGTGACAGGGGAAATTTGTGAGCGGCCTCCACCT | SEQ ID 3 |
| AAACGACTTACTAGGGAAGCTATGCGAAATTATTTAAAAGAGCGAGGGGATCAAACAGT | |
| ACTTATTCTTCATGCAAAAGTTGCACAGAAGTCATATGGAAATGAAAAAAGGTTTTTTTGC | |
| CCACCTCCTTGTGTATATCCTTATGGGCAGTGGATGGAAGAAAAAAAAAGAACAAATGGAA | |
| CGCGATGGTTGTTCTGAACAAGAGTCTCAACCGTGTGCATTTATTGGGATAGGAAATAGT | |
| GACCAAGAAATGCAGCAGCTAAACTTGGAAGGAAAGACTATTGCACAGCCAAAACATTG | |
| TATATATCTGACTA | |
| PR2-5A Human mRNA for E6-AP isoform-I | |
| GATTCGGAGAATGATGGAGACATTTCAGCAACTTATTACTTATAAAGTCATAAGCAATGA | SEQ ID 4 |
| ATTTAACAGTCGAAATCTAGTGAATGATGATGATGCCATTGTTGCTGCTTCGAAGTGCTTG | |
| AAAATGGTTTACTATGCAAATGTAGTGGGAGGGGAAGTGGACACAAATCACAATGAAGA | |
| AGATGATGAAGAGCCCATCCCTGAGTCCAGCGAGCTGACACTTCAGGGAACTTTTGGGAG | |
| AAGAAAGAAGAAACAAGAAAGGTTCCTCGAGTGGACCCCCTGGAAACTGAACTTGGTGTT | |
| AAAACCCTGGATTGTCGAAAACCACTTATCCCTTTTGAAGAGTTTATTAATGAACCACTGA | |
| ATGAGGTTCTAGAAATGGATAAGATTTACTTTT | |
| PR2-20 3 Human mRNA for TPRD | |
| GGAATATGTCTTACCCCTGACTGTGAAGGTGTCATTTCTAAGATTATCATCTTCAGCAGTG | SEQ ID 5 |
| GTGGTGAAGTTAAATGTGAATTTGAACACAAGGTCATAAAAGAAAAGGTTCCTCCAAGAC | |
| CTATTCTGAAACAGAAATGTTCTAGCCTAGAGAAACTAAGACTGAAAGAAGACAAAAAAT | |
| TGAAGAGAAAGATCCAAAAAAAAGAAGCAAAAAGTTAGCACAAGAAAGAATGGAGGA | |
| GGACTTAAGAGAAAGTAATCCACCCAAAAATGAAGACAGAAAGAAACTGTAGACAATGT | |
| TCAAGCGTTGTCAGTTCCTTGATGACAGAATTCTACAGTGTATAAAGCAGTATGCTTGACA | |
| GGATTAAATCCGGCATACAGAATACAGCCATGCTTCTAAAGAATTGTTT | |
| PR2-1B Unknown | |
| GGGAAGCAGAAGGATTTGGAGTTTCTTTTTAAAGTGATTCCTTCCTTTCCCCTTTCATTTTT | SEQ ID 6 |
| CCACTGTGGGTGTTATTATCCTGACAATTTGTCATACATTTCCTGTCTTTAAAAAATAACTG | |
| TATACTAAGCAAAACTCAGGTCTTAAAAATAAATATGAATTTAGATTCCATACATCGATTA | |
| ATTGAGGAAACACAGATCTTCCAGATGCAACAATCATCAATTAAGTCACGCGGCGACATG | |
| GTGGCCCCTGCCTCACCCCCCCAGGGATACCTGTAATACCTGCTTCCCACTTCATGGGCTAC | |
| AATCTCATGCTGTCACAATTTCTGTGCTCACTCATATAACACCACAAATGGGATATTTGTG | |
| AAGAACTTCGCTGCGGAGCT | |
| PR2-2 Unknown | |
| AGGGACAGCTCTTGCATCGAGACCCCTTCACTGTCATCTGTGGCCGAAAGAAGTGCCTTCG | SEQ ID 7 |
| CCATGTCTTTCTCTTCGAGCATCTCCTCCTGTTCAGCAAGCTCAAGGGCCCTGAAGGGGGG | |
| TCAGAGATGTTTGTTTACAAGCAGGCCTTTAAGACTGCTGATATGGGGCTGACAGAAAAC | |
| ATCGGGGACAGCGGACTCTGCTTTGAGTTGTGGTTTCGGCGGCGGCGTGCACGAGAGGCA | |
| TACACTCTGCAGGCAACCTCACCAGAGATCAAACTCAAGTGGACAAGTTCTATTGCCCAG | |
| CTGCTGTGGAGACAGGCAAGCCCACAACAAGGAGCTCCGAGTGCAGCAGATGGTGTCATG | |
| GCATTGGGAATAAACCCTTCTGGACATAAAGCCCTTGGGGAGCGA | |
| Pr3-41 Unknown | |
| GCGGCGGCGGCCCCTCGCAGCAGCTGGCCGGCGGGCCCCCCCAGCA | SEQ ID 8 |
| GTTCGCGCTCTCCAACTCCGCGGCCATCCGGGCCGAGATCCAGCGCT | |
| TCGAGTCCGTGCATCCCAATATCTACGCCATCTACGACCTGATCGAGC | |
| GCATCGAGGATTTGGCGCTGCAGAACCAGATCCGGGAGCACGTCATC | |
| TCCATCGAGGACTCGTTTGTGAACAGCCAGGAGTGGACGCTGAGCCG | |
| CTCCGTACCGGAGCTTAAAGTGGGCATAGTGGGGAACCTGTCTAGCG | |
| GGAAGTCAAGCCCTGGTGCACCGCTATCTGACGGGGACCTATGTCCA | |
| GGAGGAGTCCCCTGAAGGGGGGCGGTTTAAGAAGGAGATTGTGGTG | |
| GATGGCAGAGTTCCTGCTGTGATC | |
| Pr3-42 Unknown | |
| GGCTGGCAGTAGAGGTGACCGAGGCGGTGGCGGCGGAGGCGGCACC | SEQ ID 9 |
| GATTGCTGTGTCGGCCCCAGTGCGGCCGAAGTCGCGGTAGAGCGTAG | |
| CCCCACGCCCCTCCCCCGTCCGCGCCCTCCCTCTTTCCCTGGGGATG | |
| GAGAAGGCGACGGTTCCTGGTGGCGGCGGCGACGGCTTGCAGAAGG | |
| AGAAGGGAGCCCCCCGGCGGTGGCGGCTTGTGGCGGGCCCCCCCGC | |
| GGCGGCGGAGGTCGGCGGCGGCGTTGGCGGCAGCAGCAGAGCTCGC | |
| TCGGCCTCGTCTCCTCGTGGGATGGTGCGAGTCTGCGACCTGCTCCT | |
| GAAGAAGAAGCCGCCGCAGCAGCAGCACCACAAGGCCAAGCGTAAC | |
| CGGACTTGCCACCCCCCAGCAGCAGCGAAAC | |
| Pr3-90 Unknown | |
| GCGGCGGCGGCCCCTCGCAGCAGCTGGCCGGCGGGCCCCCCCAGCA | SEQ ID 10 |
| GTTCGCGCTCTCCAACTCCGCGGCCATCCGGGCCGAGATCCAGCGCT | |
| TCGAGTCCGTGCATCCCAATATCTACGCCATCTACGACCTGATCGAGC | |
| GCATCGAGGATTTGGCGCTGCAGAACCAGATGGGGGAGCACGTCATC | |
| TCCATCGAGGACTCGTTTGTGAACAGCCAGGAGTGGACGCTTGAGCC | |
| GCTCCGTACCGGAGCTTAAAGTGGGCATAGTGGGGAACCTGTCTAGC | |
| GGGAAGTCAGCCCTGGTGCACCGCTATCTGACGGGGACGTATGTCCA | |
| GGAGGAGTCCCTGAAGGGGGGCGGTTAAGAAGGAGATTGTGGTGGA | |
| TGGCAGAGTTCCTGCTGC | |
| Pr3-93 Unknown | |
| ATTATGAAGTAACTGAACTTTTGGTCAAGCATGGTGCGTGTGTAAATG | SEQ ID 11 |
| CAATGGACTTGTGGCAATTCACTCCTCTTCATGAGGCAGCTTCTAAGA | |
| ACAGGGTTGAAGTATGTTCTCTTCTCTTAAGTTATGGTGCAGACCGAA | |
| CACTGCTCAATTGTCACAATAAAAGTGCTATAGACTTGGCTCCCACAC | |
| CACAGTTAAAAGAAAGATTAGCATATGAATTTAAAGGCCACTCGTTGC | |
| TGCAAGCTGCACGAGAAGCTGATGTTACTCGAATCAAAAAACATCTCT | |
| CTCTGGAAATGGTGAATTTCAAGCATCCTCAAACACATGAAACAGCAT | |
| TGCATTGTGCTGCTGCATCTCCATATCCCAAAAGAAAGCAAATATGTG | |
| AACTGTTGCTAGAAAAGC | |
| Pr3-104 Unknown | |
| CCTCAGCATACCCACCGAGCAGCTGCCAGCCTGGGCTGAGGGTGGGC | SEQ ID 12 |
| ATGAGGCAGGAGTCAGCACTTGGACCTAGGGATGTGAGGTTTTCTGT | |
| GCCCCAAGTTTGTGGGAAGGTGGGCACTACTGCTGGGCCCACAGACA | |
| CAGCCAGCTGGCAAAAGGGAGGTCTAGCCCAGCAGAGAGATGAGGA | |
| CATTTTGCTTCTCCTTCATGCCCACAGCATGAGCTGAGCTTCTGCTTT | |
| GCTGGAAATGAAATAAAGTTGGTATGAATTGTGCCAAGGCCTCCCCA | |
| GTTGTCATCCTGCCTCTTGTTGCCCTCCCTTGTCCTTGCCCCCCACCC | |
| CACACCCATGCCCCTGTTTCCTTACAGATTTTGATATTGTCTAATGTG | |
| TAATAGAACCAGCCGAGTCCCA | |
| Pr3-113 Unknown | |
| CTTACCTCATTTCTGAATGTGCATTTCCAGCCTTCTTGCTCTCAGAGC | SEQ ID 13 |
| TATTGTTCAAGCAGAAAACAAGGTGCTTTTATTACA | |
| Pr3-122 Unknown | |
| GAGAGAACTAGTCTCGAGTTTTTTTTTTATTCTTCTATATTCTATGAAT | SEQ ID 14 |
| ATGGTGCTGTCGTGTCATTTMTTATTATAATATATGTGAACTGCTGG | |
| AGGTAAA | |
| Pr3-124 Unknown | |
| TCGATGCTTAGTGACTTAACAATCAGGCCTTAATTGAAACACAGACAC | SEQ ID 15 |
| ACATTGTTATTGACAGTGTAGAAATACTGACTCATAGAAAAATTCACC | |
| CATATTTAGTTAGCAGACTAACAGGAACAGCAGCAGCAGCAGCAGCT | |
| GGTCATGCTTCTGTGTGTTGCTAGCAACAAGAAACCATGACAGCAAG | |
| GCCCCAAACAGGAACCTCCTGCATTTTGTCATCTGTGATGAGGCACAG | |
| TTGATGCTGGGGATTAATGAGCCTGAAGATATAAAGCAGTGTTTACC | |
| ACTGGAAAATGTCTCCTACACTAAAAGCAGAGGTAAGTATCAATGCA | |
| AACCGAGTGCAGCTATAAAGCCTTGATTTCTCTGGAAATTATGTACAA | |
| ACTAATACAAATAATCTCATTACTTGAAAC | |
| Pr3-133 Unknown | |
| GCTACGGCTGCTCCGGAGCTGGTGGCGCCGCGATAGGAGAGCCGAT | SEQ ID 16 |
| GGCCAAGTGGGGTGAGGGAGACCCACGCTGGATCGTGGAGGAGCGG | |
| GCGGACGGCACCAACGTCAACAACTGGGACTGGACGGAGAGAGATGC | |
| TTCAAATTGGTCCACGGATAAGCTGAAAACACTGTTCTTGGCAGTGCA | |
| GGTTCAAAATGAAGAAGTCAAGTGTGAGGTGACGGAAGTGAGTAAGC | |
| TTGATGGAGAGTCATCCATTAACAATCGCAAAGGGAAACTTATCTTCT | |
| TTTATGAATGGAGCGTCAAACTAAACTGGACAGGTACTTCTAAGTCAG | |
| GAGTACAGTACAAAGGACATGAGGAGATCCCCAATTTGTCTGATGAA | |
| AAC | |
| Pr3-140 Unknown | |
| CATTACCTTACAGTGTAAACAGGAGTCTAATTTGTATCAATACTATGT | SEQ ID 17 |
| TTTGGTTGTAATATTCAGTTCACTCACCCAATGTACACCAATGAAAT | |
| AAAAGAAGCATTTAAAAGGAA | |
| Pr3-147 Unknown | |
| GGCGTGTGGGTCTCGGAGCGTTGCTCACAGAACAGAGTAGAGGCGGC | SEQ ID 18 |
| GGCGGCGGCGGCCGGACCCAGACTGGTAGTGAGGCGTTGGACCCCG | |
| AGCCGCTGCAATGCCGCTGGAGCTGGAGCTGTGTCCCGGGCGCTGG | |
| GTGGGCGGGCAACACCCGTGCTTCATCATTGNCGAGATCGGCCAGAA | |
| CCACCAGGGCGACCTGGACGTAGCCAAGCGCATGATCCGCATGGCCA | |
| AGGAGTGTGGGGCTGATTGTGCCAAGTTCCAGAAGAGTGAGCTAGAA | |
| TTCAAGTTTAATCGGAAAGCCTTGGACAGGCCATACACCTCGAAGCA | |
| TTCGTGGGGGAAGACGTACGGGGAGCACAAACGACATCTGGAGTTCA | |
| GCCATGACCAGTCAGGGAGCTGAGAGGTCC | |
| Pr3-14S Unknown | |
| GACGGACGGAGACCGGAGATGTTTTCAAGCCCGGCTCCGGCGGCTTT | SEQ ID 19 |
| ACAGGCGGCTGCAGCGGCGACGAAGACAACGACAGCGACGGCTACG | |
| CCGAAGCACTCGAACCGGGGGTGAAGCCTCCTGCGCCGGCCTTGCCT | |
| CGGATCCAGGATGAGAAGACTGATAAAAGAAGAAGCTAGCTGAACAG | |
| CTGTAAAATGCCCAAATCTGGGTTCACAAAACCAATTCAGAGTGAAAA | |
| TTCTGACAGTGAGAGGAATATGGTAGAGAAACCATATGGAAGAAAGA | |
| GTAAAGACAAGATTGCATCCTACAGCAAAACTGCAAAAATTGAACGA | |
| AGTGATGTGAGCAAGGAGATGAAAGAGAAATCATCCATGAAACCGTA | |
| AACTTCCTTTC | |
| Pr3-162 Unknown | |
| GCAGGAGGGGCCTTGCCAGCTTCCGCCGCCGCGTCGTTTCAGGACCCGGACGGCGGA | SEQ ID 20 |
| TTCGCGCTGCCTCCGCCGCCGCGGOGCAGCCGGGGGGCAGGGAGCCCAGCGAGGGGC | |
| GCGCGTGGGCGCGGCCATGGGACTGCGCCGGATCCGGTGACAGCAGGGAGCCAAGCG | |
| GCCGGGCCCTGAGCGCGTGTTCTCCGGGGGGCCTCGCCCTCCTGCTCGCGGGGCCGG | |
| GGCTCGTGCTCCGGTTGCTGGCGCTGTTGCTGGCTGTGGCGGCGGCCAGGATCATGT | |
| CGGGTCGCCGCTGCGCCGGCGGGGGAGCGGCTGCGCGAGCGCCGCGGCCGAGGCCGT | |
| GGAGCCGGCCGCCGAAGCTGTTCGAGGCGTGCCGAACGGGGACGTGGAACGAGTAAG | |
| AGGCTG | |
| Pr3-180 Unknown | |
| GCCAACTCAGTCCAGCAGAACAAAATGTAGCTGCCATTCTTGGAGTC | SEQ ID 21 |
| TCTGAAAGCTTTATTGGGAAGAAAGCATCAGGCCAAGCCATCGGAAA | |
| GAAGGTGGACAAGAACGTTGTCAACAGGCTATATCTGTCTTTTGTTCT | |
| TTATACCTTGCTCAAAGAGACCAACATTTGGACTGTATCTGAAAAATT | |
| TAATATGCCTCGAGGATATATACAAAATCTTCTCACTGGAACTGCCTC | |
| ATTCTCATCTTGTGTGTTACATTTCTGTGAGGAGCTTGAGGGAGTTTT | |
| GGGTTTACAGAGGCCTTTrGGTAGAACTTACCAAGAAGCTGACTACT | |
| GTGTAAAGGGCAGAATTAATCCCTCTATGGGAAGTTCTNGGAGTTTTA | |
| GAGGGTCGAGCAAAACAGTTTTTCAGNGCCNGGTACCAAAAGTCTAA | |
| TGCCTTAGCTAAGGAAACCCTGAANGNTTCTANGGNCAATTGGTCNTT | |
| TTTTAAGACCCCAAGCCAGCAAATTGTTTATNCAAAAATCTNTTCNTN | |
| AAAACCAAACCTCAAAANGGNNAAAAGTCCNAAATGCTTTTNTTCCCG | |
| GGGGNGGGGGTTNTTCCCGGCAAACNGAANTTTTTGNGGGAANTTTT | |
| TTTTAATTTTTTTNG | |
| Pr3-187 unknown | |
| GGGAGGCGGGGGCAGCGTTAAGTGAGAAAGGAAAAAAGACAACGAGGAAAAAGGAGG | SEQ ID 22 |
| TGTCCGGGTAGGGCAACGCGGCGACACCCGAGGCCTGGTGGTGGCGGCGGATCGAGA | |
| TATTCAAGGCTGAAGCAGCTACGGAACGGCAGCGGCGGCGGTCGGACAAACTGACTG | |
| ACCGAGCCGGGTGGTGGCGGGAGCAGGGGGAGCAGCCGGAACGATGCCGGCCGTGAG | |
| CCTCCCGCCCAAGGAGAATGCGCTCTTCAAGCGGATCTTGAGGTGTTATGAACATAA | |
| ACAGTATAGAAATGGATTGAAATTCTGTAAACAAATACTTTCTAATGCCAAATTTGC | |
| AGAGCATGGAGAAACCTTGGCTATGAAAGGATTAACATTGAACTGTTTGGGGAAAAA | |
| GGAAGAACTTATGAATTGGTTCCTAGAGGTTTGAGAAATGACTTGAAGAGTCATGTG | |
| TGTTGGCCACGTTTATGGCCTTTTTCAAGGTCANACAAGAAGTNTGATGAANNCCTT | |
| AANTGTTACAGAAATGCCTAAATGGGATAAGACATCTTAAATTTTAAGGGNCTTTCT | |
| TCTACAANTCAATCCAAACTNGNGGNTTCCNGGAAGCAGGTTTNANTTCTTCANTTN | |
| CNCCTCCCAAAGCATTATGNT | |
| Pr3-194 Unknown | |
| CGGTGGCGGCGGAGGCGGCACCGATTGCTGTGTCGGCCCCAGTGCGGCCGAAGTCGC | SEQ ID 23 |
| GGTAGAGCGTAGCCCCACGCCCCTCCCCCGTCCGCGCCCTCCCTCTTTCCCTGGGGA | |
| TGGAGAAGGCGACGGTTCCGGTGGCGGCGGCGACGGCTGCAGAAGGAGAAGGGAGCC | |
| CCCCGGCGGTGGCGGCTGTGGCGGGCCCCCCCGCGGCGGCGGAGGTCGGCGGCGGCG | |
| TTGGCGGCAGCAGCAGAGCTGGCTCGGCCTCGTCTCCTCGTGGGATGGTGCGAGTCT | |
| GCGACCTGCTCCTGAAGAAGAAGCCGCCGCAGCAGCAGCACCACAAGGCCAAGCGTA | |
| ACCGGACTTGCCGACCCCCCAGCAGCAGCGAAAGCAGCAGCGACAGCGAGAACAGCG | |
| GCGGCGGTGGAGGGGGCGGTGGAGCGGAAGTGGCGGCGGCGGCACCAGCANTAACAA | |
| CAGCGAGGAAANAAAGGACACACACGAGGAANAGAGGTTNTGAGGGGAGTTTTATTT | |
| GGNTCAGATTATTGGAAANTCAANCTTGNAAACTTCCAGGTNNTCTATAANGTCNNT | |
| TGTNGNGCATACNTANGAANTANNCCAAAANNAGNTTTNATGGGAGTTTTACNAAAC | |
| NCAGTTTGGATC | |
| Pr3-199 Unknown | |
| CTNNGTTTTTTTTTTTTTTTTTTTCCAGACTCTTCTGTTCTTTTATATCTCAGAAAAG | SEQ ID 24 |
| GATTGGGTTTTCAGGTTGCAAAATCTTTTCCAGCTCTGCATAGGTAGGTAGCATCTC | |
| ACTGAGGAATGGAGTATTTACCACCTATTGTTCTGTNCCAGTCTAGTAGAGCTTTAG | |
| CAAAANCTACAGGCAACAAATTCTATTTTTAACATCCTGTTACACAAACAAATATGC | |
| TGAGTATGCACACAAATAAATGGTGAAAGAGGCNCAAAGAAGTGAAAACAATCGTGC | |
| ATGGTAGGAATATTTGAATTGTNTTACATGTCCTTTAATATTGNTTTAACAGTNATA | |
| TTTTTACATTTTCAATTGGGAATGAAAAGCATGTCTGTGTTCGAATAATTTTTCATCG | |
| NNCNCTCATTTTTTTGATTCCCNANCTAATGAGNAGAAANCAGTGATGATTGCAAAA | |
| TGTTTCCCNCCCTNAAGGAATNCNCGTNNGAATTCTTGCAGNTCCTGGAGANCTCCN | |
| TANTTTANGNGNTATATAGGTANNGATCTATACTCCCTCGGGGGGTCTTAGCCTNNC | |
| GNNCCTNCCTTCNNTCTCACNANCATTGTTNTCTANNGCNNCTCANNTAANTNCTN | |
| CAGGCCCNCAANTGNNTATNNANCCNCNNNCNTNTC | |
| Pr3-201 Unknown | |
| CCCGGAACCTGCAAGGCCTGGTGTGGGACCCACACAACCGTAGGAGA | SEQ ID 25 |
| CAGGTCCTGAATACCCGGGCCCAAGAGCCCAAGCTGTGCTGGCCTCA | |
| GGGTTTCTCCTGGAGTCACCGAGCCGTGGTCCACTTCGTCGTGCCTG | |
| TGAAGAACCAGGCACGGTGGGTACAGCAATTCATCAAAGACATGGAA | |
| AACCTGTTCCAGGTCACCGGTGACCCACACTTCAACATCGTCATCACT | |
| GACTATAGCAGTGAGGACATGGATGTTGAGATGGCACTGAAGAGGTC | |
| CAAGCTGCGGAGCTACCAGTACGTGAAGCTAAGTGGAAACTTTGAAC | |
| GCTCAGCTGGACTTCAGGCTGGCATAGACCTCGTGAAGGACCCGCAC | |
| AGCATCATCTTCCTCTGTGACCTCCACATCACTTCCCACTTGGAGTCA | |
| TNGATGGCATTCGGAACACTTGTGTGGAGGGAAAAGAAGGGCTTTTG | |
| CCCCCTGGTGATAAGGTTGNNTTGGGGGCNCCCCCAANGGCTGAGGC | |
| TCGGGAGGGAAAAGGGTTGGGNNTTGGATTACAATTTNCCTGANAN | |
| GATGGGGGCNTAACCAAAAGGANTCCAAANCCTGGGNGGGAAAAANG | |
| GNACTTTTNNAGGAATTTCAANGCN | |
| Pr3-202 Unknown | |
| GTGAGATGAATGTTCCCCCTTCAATTCTCCTTATTTGCCAAATATTTT | SEQ ID 26 |
| CATTTCCTTTTGTCATTATAGAAAATAAAACCATGCATCACA | |
| Pr3-205 Unknown | |
| AGGAACCAAAGAAGACATGGTCCCTGTCCTCATGGTTCAGACAGGGAGGCAGACATT | SEQ ID 27 |
| AAACAACTAATTATCAGTTATTCAATTA | |
| Pr3-208 Unknown | |
| GCGACTCGGGGACCTGGAGCTGACGCCTAGACACTTGTATTAGCTTT | SEQ ID 28 |
| AATAGAAGAGAAATGGAGGAGCCATAGAATATTAAGGATGAATTCAG | |
| GAAGGCGTGAGAGCATGGAAAACTTGCCTGCTCTCTACACTATTTTCC | |
| AAGGAGAGGTTGCTATGGTGACAGACTATGGGGCCTTTATCAAAATC | |
| CCAGGCTGTCGGAAGCAAGGTCTGGTCCATCGAACTCATATGTGATC | |
| CTGTCGGGTGGATAAGCCCTCTGAGATAGTAGATGTTGGAGATAAAG | |
| TGTGGGTGAAGCTTATTGGCCGAGAGATGAAAAATGATAGAATAAAA | |
| GTATCCGTCTCCATGAAGGTTGTCAATCAAGGGGACTGGGAAAGACC | |
| TTGATCGCAACAATGTTATCATTGAGCAAGAAGAGANGCGGAGGCGA | |
| TCCTTCCAGGATTACACTGGGCAGNAAGATCACGCTTGAGGCTTGTCT | |
| TGACCCTACCTCAANAAGNGNGGNTGTAAAGGGCCCTTTGCAAAAAA | |
| TGGTTATGCANCNGGGGGAATTAAACTTTTTTTCCNTTGGGAAAGGAA | |
| AGGAAAGCCAATCCCCANTTTGNAAACCTNCCTCAGGAATCTTTTAAA | |
| NAAAGAGGGAAAAAAAANAACCN | |
| Pr3-213 Unknown | |
| CTGTCATGGCTGCTCCTGTACGTAGTCACGGTCTTGTGCTCTAAGGAA | SEQ ID 29 |
| AACGACAGCACGTGTTCTTTTTCACTAGTAGAAGTGACGTTGGTTTCA | |
| TGTTGACAACTTTGAAGGCATTTGGAAGTGTTTCAGTGGAGAACAAAA | |
| TGAATAACAAAGCGGGCTCGTTTTTCTGGAAGCTTAGACAATTCAGTA | |
| CATTAGTTTCAACAAGCAGAACTATGAGGCTATGTTGTTTGGGACTTT | |
| GCAAACCAAAAATAGTTCATTCAAACTGGAACATTTTAAATAACTTTC | |
| ATAACAGAATGCAATCAACTGATATCATTAGATATCTCTTTCAGGATG | |
| CATTCATTTTTAAATCAGATGTTGGCTTTCAAACAAAGGGCATAAGCC | |
| TCTACAGCCCTTAGAATTGAAGAC | |
| Pr3-214 Unknown | |
| GTATGGCGGCGTCAAAGGTGAAGCAGGACATGCCTCCGCCGGGGGG | SEQ ID 30 |
| CTATGGGCCCATCGACTACAAACGGAACTTGCCGCGTCGAGGACTGT | |
| CGGGCTACAGGATGCTGGCCATAGGGATTGGAACCCTGATCTACGGG | |
| CACTGGAGCATAATGAAGTGGAACCGTGAGCGCAGGCGCCTACAAAT | |
| CGAGGACTTCGAGGCTCGCATCGCGCTGTTGGCACTGTTACAGGCAG | |
| AAACCGACCGGAGGACCTTGCAGATGCTTCGGGAGAACCTGGAGGAG | |
| GAGGCCATCATCATGAAGQACGTGCCCGACTGGAAGGTGGGGGAGT | |
| CTGTGTTCCACACAACCCGCTGGGTGGCCCCCTTGATCGGGGAGCTG | |
| TACGGCTTGCGCACGACAGAGOAGGCTCTTCATGCGAGCC | |
| Pr3-2 Homo sapiens geminin mRNA | |
| GCAGGGCTTTACTGCAGAGCGCGCCGGGCACTCCAGCGACCGTGGG | SEQ ID 31 |
| GATCAGCGTAGGTGAGCTGTGGCCTTTTGCGAGGTGCTGGAGCGATA | |
| GCTACGTGCGTTGGCTACGAGGATTGAGCGTCTCCACCCATCTTCTGT | |
| GCTTCAGCATCTACATAATGAATCCCAGTATGAAGCAGAACAAGAAG | |
| AAATGAAAGAGAATATAAAGAATAGTTCTGTCCCAAGAAGAACTCTGA | |
| AGATGATTCAGCCTTCTGCATCTGGATCTCTTGTTGGAAGAGAAAATG | |
| AGCTGTCCOCAGGCTTGTCCAAAAGGAAACATCGGAATGACCACTTA | |
| ACATCTACAAGTTCCAGCCCTGGGGTTATTGTCCCAGAATGTAGTGAA | |
| AATAAAATCTTGGAGGAGTACGCAGGA | |
| Pr3-8 Homo sapiens scaffold attachment factor A | |
| GCGAACTCGGTGAAAGGAATTGGCGCCGTTCGACACCAGGCGGATCC | SEQ ID 32 |
| GCTCTGCAGCACGAACCGATCTCCAGCCGCAGCCGCAGCCGCCGCCC | |
| GGGCCGAGGAGCAGCCGCAGCAGCCGGACCAGTGGCCGAGTGAGCG | |
| GAGCCGAGTTTGAGGCAGCGCCTAGCGGTGAATCGGGGCCCTCACCA | |
| TGAGTTCCTCGCCTGTTAATGTAAAAAAGCTGAAGGTGTCGGAGCTG | |
| AAAGAGGAGGTCAAGAAGCGACGCCTTTCTGACAAGGGTCTCAAGGC | |
| CGAGCTCATGGAGCGACTCCAGGCTGCGCTGGACGACGAGGAGGCC | |
| GGGGGCCGCCCCGCCATGGAGCCCGGGAACGGCAGCCTAGACCTGG | |
| GCGGGGATTCCGCTGGGA | |
| Pr3-11 Homo sapiens ribosomal protein L32 | |
| CCTACGGAGGTGGCAGCCATCTCCTTCTCGGCATCATGGCCGCCCTC | SEQ ID 33 |
| AGACCCCTTGTGAAGCCCAAGATCGTCAAAAAGAGAACCAAGAAGTT | |
| CATCCGGCAGCAGTCAGACCGATATGTCAAAATTAAGCGTAACTGGC | |
| GGAAACCCAGAGGCATTGACAACAGGGTTCGTAGAAGATTCAAGGGC | |
| CAGATCTTGATGCCCAACATTGGTTATGGAAGCAACAAAAAAACAAA | |
| GCACATGCTGCCCAGTGGCTTCCGGAAGTTCCTGGTCCACAACGTCA | |
| AGGAGCTGGAAAGTGCTGCTGATGTGCAACAAATCTTACTGTGCCGA | |
| GATGGCTNACAATGTTTCTTCAAGACCGCAAAGCC | |
| Pr3-13 Homo sapiens glutamyl-prolyl-tRNA synthetase | |
| GTCGGGTACGCGCACACGTTGCATCTTCTTCCTTTCGCGGGGTCCTC | SEQ ID 34 |
| CGTAGTTCTGGCACGAGCCAGGGGTACTGACAGGTGGACCAGCGGAC | |
| TGGTGGAGATGGCGACGCTCTCTCTGACCGTGAATTCAGGAGACCCT | |
| CCGCTAGGAGCTTTGCTGGCAGTAGAACACGTGAAAGACGATGTCAG | |
| CATTTCCGTTGAAGAAGGGAAAGAGAATATTCTTCATGTTTCTGAAAA | |
| TGTGATATTCACAGATGTGAATTCTATACTTCGCTACTTGGCTAGAGT | |
| TGCAACTACAGCTGGGTTATATGGCTCTAATCTGATGGAACATACTGA | |
| GATTGATCACTGGTTGGAGTC | |
| Pr3-30 Homo sapiens geminin mRNA (mutation at nt 220) | |
| GCGGAGTTAGCAGGGCTTTACTGGAGAGCGCGCCGGGCACTCCAGCG | SEQ ID 35 |
| ACCGTGGGGATCAGCGTAGGTGAGCTGTGGCCTTTTGCGAGGTGCTG | |
| CAGCCATAGCTACGTGCGTTCGCTACGAGGATTGAGCGTCTCCACCC | |
| ATCTTCTGTGCTTCACCATCTACATAATGAATCCCAGTATGAAGCAGA | |
| AACAAGAAGAAATGAAAGAGAATATAAAGACTAGTTCTGTCCCAAGA | |
| AGAACTCTGAAGATGATTCAGCGTTCTGCATCTGGATCTCTTGTTGGA | |
| AGAGAAAATGAGCTGTCCGCAGGCTTGTCCAAAAGGAAACATCGGAA | |
| TGACCACTTAACATCTACAACTTCCAGCCTGGGGGTTATTGTCCCAGA | |
| ATTCTAGTGAAAATAAAAATTTNGNNGGGAGTCACCCANGGAGTATTT | |
| TTGATCTTATGATTAAAGGAAAATCCATCTTTTAATATTGAAGGGGAA | |
| GNGGGCAGAAAAACGGAAAAGGGGNCCTTTNTGAAGCACTTAAGGGA | |
| AAATGAGNAAACTTCATAAAGNAAATTGACCAAANGGACAATTGAAA | |
| ATGGCCCGCTGAAAAAGGAAAATAAAGACTGGCNNNAAGTAGCAAAA | |
| CATGTCCNGGTTTTTG | |
| Pr3-43 Homo sapiens DNA-binding protein (HRC1) mRNA | |
| (5′ end of the clone corresponds to the beginning of exon 2 of | |
| HRC1) | |
| CAGGCATGTTGTTGGGACTGGCGGCCATGGAGCTGAAGGTGTGGGTG | SEQ ID 36 |
| GATGGCATCCAGCGTGTGGTCTGTGGGGTCTCAGAGCAGACCACCTG | |
| CCAGGAAGTGGTCATCGCACTAGCCCAAGCAATAGGCCAGACTGGCC | |
| GGTTTGTGCTTGTGCAGCGGCTTCGGGAGAAGGAGCGGCAGTTGCTG | |
| GCACAAGAGTGTCCAGTGGGCGCCCAGGCCACCTGCGGACAGTTTGC | |
| CAGCGATGTGCAGTTTGTCCTGAGGGGCACAGGGGCCAGCCTAGCTG | |
| GGAGGCCCTCCTCAGACAGCTGTGCACCCCCGGAACGCTGCCTAATT | |
| CGTGCCAGCCTCCCTGTAAAGCCACGGGCTTGCGCTTGGGCTGTGAG | |
| CCCCGCAAAACACTGACCCCGAGCCAGCCC | |
| Pr3-49 Homo sapiens vesicle docking protein p115 mRNA | |
| GCGAGTTGGAGGCGGTGGAGCCAGCAGTAGGAGTGTGTAGACATGCG | SEQ ID 37 |
| GGATTGGGGGCCAGGCCCTGCGGAGGGCGGGGGAAGTTGTCTTCTTT | |
| TTTTTCCGGAGGGGCCGGTAAACCTGGTGGCTGAACGGCAAGATGAA | |
| TTTCCTCCGCGGGGTAATGGGGGGTCAGAGTGCCGGACCCCAGCACA | |
| CAGAAGCCGAGACGATTCAAAAGCTTTGTGACAGAGTAGCTTGATCT | |
| ACTTTATTGGATGATCGAAGAAATGCTGTTCGTGCTCTCAAATCATTA | |
| TCTAAGAAATACGGCTTGGAAGTGGGTATACAAGCTATGGAACATCTT | |
| ATTCATGTTTTACAAACAGATCGTTCANATTCTGAAATTATAGGTATG | |
| CTTTGGACACACTATATAATNNATATCTAA | |
| Pr3-101 Homo sapiens upstream transcription factor, c-fos | |
| interacting (USF2) | |
| ACATGCTGGACCCGGGTCTGGATCCCGCTGCCTCGGCCACCGCTGCT | SEQ ID 38 |
| GCCGCGGCCAGCCACGACAAGGGACCCGAGGCGGAGGAGGGCGTCG | |
| AGCTGCAGGAAGGCGGGGACGGCCCAGGAGCGGAGGAGCAGACAGC | |
| GGTGGCCATCACCAGGGTGCAGCAGGCGGCGTTCGGCGACCACAACA | |
| TCCAGTACCAGTTCCGCACAGAGACAAATGGAGGACAGGTGACATAC | |
| CGCGTAGTCCAGGTGACTGATGGTCAGCTGGACGGCCAGGGCGACAC | |
| AGCTGGCGCCGTCAGCGTCGTGTCCACCGCTGCTTCGCGGGGGGGCA | |
| AGCAGGCTGTGACCAGGTG | |
| GGTGTGC | |
| Pr3-109 Homo sapiens DNA-binding protein (HRC1) mRNA (Type I | |
| transcript) | |
| GTCGGGGTGGGGCGTTCCCATGCCGGCGGCCGCGGGGCCTGGCGTG | SEQ ID 39 |
| CGGGCGCCTCCGCGCCGCCCGGGGAGGGGGCAGTGTCCTCCGAGCC | |
| AGGACAGGCATGTTGTTGGGACTGGCGGCCATGGAGCTGAAGGTGTG | |
| GGTGGATGGCATCCAGCTGTGTGGTGNTGTGGGGTCTCAGAGCAGAC | |
| AGCTGCCAGGAAGTGGTCATCGCACTAGCCCAAGCAATAGGCCAGAC | |
| TGGCCGCTTTGTGCTTGTGCAGCGGCTTCGGGAGAAGGAGCGGCAGT | |
| TGCTTGCCACAAGAGTGTCCAAGTGGGCGCCCAGGCCACCTGCGGAC | |
| AGTTTGCCAGCGATGTCCAGTTTGTCGTGAGGCGCACAGGGCCCAGC | |
| CTAGCTGGGAGGCCTTCTAGACAGGTGC | |
| Pr3-111 Homo sapiens proteasome sub-unit HSPC mRNA | |
| GAGTCGCGGCGGAAGGAGCCCGGCCGCCGCCCGCCGGCATGAGCTA | SEQ ID 40 |
| CGACCGCGCCATCACCGTCTTCTCGCCCGACGGCCACCTCTTCCAAG | |
| TGGAGTACGCGCAGGAGGCCGTCAAGAAGGGCTCGACCGCGGTTGG | |
| TGTTCGAGGAAGAGACATTGTTGTTCTTGGTGTGGAGAAGAAGTCAG | |
| TGGCCAAACTGCAGGATGAAAGAACAGTGCGGAAGATCTGTGCTTTG | |
| GATGACAACGTCTGCATGGCCTTTGCAGGCCTCACGGCCGATGCAAG | |
| GATAGTCATCAACAGGGCCCGGGTGGAGTGCCAGAGCCACCGGCTGA | |
| CTGTGGAGGACCCGGTGACTGTGGAGTACATACCCGCTACATGGCCA | |
| GTCTGAAGCAGCGTTATACGCAC | |
| Pr3-112 Homo sapiens trans-Golgi p230 mRNA | |
| GCCGAGGCCAGCCAGTGGCACCCGGAAGAAAGAGACGCGGCGGCGG | SEQ ID 41 |
| CGACGCCGAGACCCTCAGGACGAGTGTCCGGACTTGCCCACAGCCTC | |
| AAGGAGGAGACGGCGAGGCCCGGCCCCCGCTGTCCCTGGTGTAAAG | |
| AAGTCGCCGTAGCCGTCGCGGCCGGGACTCCCCGGGCTCTCGCGCTT | |
| CAGGTTTCGTTGACACTCAGGACGGTACGTACGCTTGCGCCATGTTC | |
| AAGAAACTGAAGCAAAAGATCAAGGGAGGAGCAGCAGCAGCTCCAGC | |
| AGGCGCTTGGCTCCTGCTCAGGCGTCCTCCAATTCTTCAACACCAACA | |
| AGAATGAGGAGCAGGACATCTTCATTTCAGAGCAACTTGATGAAGGT | |
| ACACCCAATAGAGAGTCAGGTGACACACAGTCTTTTGA | |
| Pr3-116 Homo sapiens ribosomal protein S14 | |
| CACCCCCATGCCCTCTGACAGCACTCGCAGGAAGGGGGGTCGCCGTG | SEQ ID 42 |
| GTCGCCGTCTGTGAACAAGATTCCTCAAAATATTTTGTGTTAATAAAT | |
| TGCCTTCATGTA | |
| Pr3-118 Homo sapiens poly (ADP-ribose) polymerase mRNA (The | |
| clone is 14 nt longer than the polymerase at 5′ end; There is | |
| a point mutation at nt 159 of the clone) | |
| GCCGCTCAGGCGCCTGCGGCTGGGTGAGCGCACGCGAGGCGGCGAG | SEQ ID 43 |
| GGGGCAGCGTGTTTCTAGGTCGTGGCGTCGGGCTTCCGGAGCTTTGC | |
| CGGCAGCTAGGGGAGGATGGCGGAGTCTTCGGATAAGCTCTATCGAG | |
| TCGAGTACGCCAAGAGCGGGCGCGCCTCTTGCAAGAAATGCAGCGAG | |
| AGCATCCCCAAGGACTCGCTCCGGATGGCCATCATGGTGCAGTCGCC | |
| CATGTTTGATGGAAAAGTCCCACACTGGTACCACTTCTCCTGCTTCTG | |
| GAAGGTGGGCCACTCCATCCGGCACCCTGACGTTGAGGTGGATGGGT | |
| TCTCTGAGCTTCGGTGGGATGATCAAGCAGAAAGTCAAGAAGACAGC | |
| GGAAGCTGGAGGAGTNCAGG | |
| Pr3-119 Honio sapiens tankyrase, TRF-interacting ankyrin- | |
| related polymerase (TNKS) mRNA, and translated products (point | |
| mutation at nt 129 of the clone) | |
| TAAAGGAAAGTATGAAATCTGCAAGCTCGTTTTAAAACATGGAGCAG | SEQ ID 44 |
| ATCCAACTAAAAAGAACAGAGATGGAAATACACCTTTGGATTTGGTAA | |
| AGGAAGGAGACACAGATATTCAGGACTTACTGAGAGGGGATGCTGCT | |
| TTGTTGGATGCTGCCAAGAAGGGCTGCCTGGCAAGAGTGCAGAAGCT | |
| CTGTACCCCAGAGAATATCAACTGCAGAGACACCCAGGGCAGAAATT | |
| CAACCCCTCTGCACCTGGCAGCAGGCTATAATAACGTGGAAGTAGCT | |
| GAATATGTTCTAGAGCATGGAGCTGATGTTAATGCCCAGGACAAGGG | |
| TGGTTTAATTCCTCTTCATAATGCGGCATCTTATGGGCATGTTGACA | |
| Pr3-128 Homo sapiens proteasome sub-unit HSPC mRNA | |
| GAAGAAACAAAAGAAAGCATCATGATGAATAAAATGTCTTTGCTTGTA | SEQ ID 45 |
| ATTTTTAAATTCATATCAATCATGGATGAGTCTCGATGTGTAGGCCTT | |
| TCCATTCCATTTATTCACACTGAGTGTCCTACAATAAACTTCCGTATTT | |
| TTA | |
| Pr3-146 Human poly(ADP-ribose) polymerase mRNA (point mutation | |
| at at 140 of the clone) | |
| GCGATGNGTATTACTGCAGTGGGGACGTCACTGCCTGGACCAAGTGT | SEQ ID 46 |
| ATGGTCAAGACACAOACACCGAACGGGAAGGAGTGGGTAAGCCCAAA | |
| GGAATTCCGAGAAATCTCTTACCTCAAGAAATTGAAGGTTAAAAAACA | |
| GGACCGTATATTCCCCCCAGAAACCAGCGCCTCCGTGGCGGCCACGC | |
| CTCCGCCCTCCACAGGCTCGGCTCCTGCTGCTGTGAACTCCTCTGCTT | |
| CAGCAGATAAGCCATTATCGAACATGAAGATCCTGACTCTCGGGAAG | |
| CTGTCGCGGAACAAGGATGAAGTGAAGGCCATGATTGAGAAACTCGG | |
| GGGGAAGTTGACGGGGACGGGCAACAAGGCTTCCCTGTGCATAAGCA | |
| CCAAAAAGGAGGTGGAAAAGATGAATAAGAAGATG | |
| Pr3-152 Homo sapiens ribosomal protein L10 | |
| AGAACANGGAGCATGTGATTGAGGCCCTGCGCAGGGCCAAGTTCAAG | SEQ ID 47 |
| TTTCGTGGCCGGCAGAAGATCGACATCTCAAAAAGTGGGGCTTCAAC | |
| AAGTTGAATGCTGATGAATTTGAAGACATGGTGGCTGAAAAGCGGCT | |
| CATCCCAGATGGCTGTGGGGTCAAGTACATCCCCAGTCGTGGCCCTC | |
| TGGACAAGTGGCGGGCCCTGCACTCATGAGGGCTTCCAATGTGCTGC | |
| CGCCCTCTTAATACTCACCAATAAATTCTACTTCCTGTCCAAAAAAAA | |
| AAA | |
| Pr3-154 Homo sapiens clone Xu-3 immunoglobulin heavy chain | |
| variable region mRNA | |
| GTCGTGGACCTCCTGCACAAGAACATGAAACACCTGTGGTTCTTCCTC | SEQ ID 48 |
| CTCCTGGTGGCAGCTCCCAGATGGGTCCTGTCCCAGGTGCAGTTACA | |
| GCAGTGGGGCGCAGGACTCTTGAAGCCTTCGGAGACCCTGTCCCTCA | |
| CCTGCGCNTGTCTATGGTGGGTCCTTAAGTGGTTATGGCTGGAGCNT | |
| GGATCCGCCAGCCCCCAGGGAAGGGGCTTGGAGTGGATTGGGGAAG | |
| TCGAGCATCGTGGGAGCGCCAATTACCAGTCGGCCCTCCAGAGTCGA | |
| GTCTCCGTATCATTGGACACGTCCAAGAACCAGGTCTCCCTGAGGCT | |
| GAACTCAGTGACCGCCGCGGAGACGGCTGTTTATNCTGTGCGAGAGG | |
| CCTAATATAAAGCAATGGCTCTATTTGGGC | |
| Pr3-155 Homo sapiens phospholipase C, gamma 1 mRNA | |
| GCCAGATCACGTGGAGCCGGGGCGCCGACAAGATCGAGGGGGCCAT | SEQ ID 49 |
| TGACATTCGTGAAATTAAGGAGATCCGCCCAGGGAAGACCTCACGGG | |
| ACTTTGATCGCTATCAAGAGGACCCAGCTTTCCGGCCGGACCAGTCA | |
| CATTGCTTTGTCATTCTCTATGGAATGGAATTTCGGCTGAAAACGCTG | |
| AGCCTGCAAGCCACATCTGAGGATGAAGTGAACATGTGGATCAAGGG | |
| CTTAACTTGGCTGATGGAGGATACATTGCAGGCACCCACACCCCTGC | |
| AGATTGAGAGGTGGCTCCGGAAGCAGTTTTACTCAGTGGATCGGAAT | |
| CGTGAGGATCGTATATCAGCCAAGGACCTGAAGAACATGCTGTCCCA | |
| GGTCAACTACCGGGTCCCAACC | |
| Pr3-157 Homo sapiens ribosomal protein S10 mRNA | |
| GTACCTTACCAATGAGGGTATCCAGTATCTCCGTGATTACCTTCATCT | SEQ ID 50 |
| GCCCCCGGAGATTGTGCCTGCCACCCTACGCCGTAGCCGTCCAGAGA | |
| CTGGCAGGCCTCGGCCTAAAGGTCTGGAGGGTGAGCGACCTGCGAG | |
| ACTCACAAGAGGGGAAGCTGACAGAGATACCTACAGACGGAGTGCTG | |
| TGCCACCTGGTGCCGACAAGAAAGCCGAGGCTGGGGCTGGGTCAGC | |
| AACCGAATTCCAGTTTAGAGGCGGATTTGGTCGTGGACGTGGTCAGC | |
| CACCTCAGTAAAATTGGAGAGGATTCTTTTGCATTGAATAAACTTACA | |
| GCCAAAAAAACCTTA | |
| Pr3-160 Homo sapiens poly(ADP-ribose) synthetase mRNA | |
| AATCCGGGCACGAGGTTCGGTGCCCTCCTTCCCTGCGAGGAATGCTC | SEQ ID 51 |
| GGGTCAGCTGGTCTTCAAGAGCGATGCCTATTACTGCACTGGGGACG | |
| TCACTGCCTGGACCAAGTGTATGGTCAAGACACAGACACCCAACCGG | |
| AAGGAGTGGGTAACCCCAAAGGAATTCCTGAGAAATCTCTTACCTCA | |
| AGAAATTGAAGGTTAAAAAACAGGACCGTATATTCCCCCCAGAAACC | |
| AGCGCCTCCGTGGCGGCCACGCCTCCGCCCTCCACAGCCTCGGCTCC | |
| TGCTGCTGTGAACTCCTCTGCTTCAGCAGATAAGCCATTATCCAACAT | |
| GAAGATGCTGACTCTCGGGAAGCTGTCCCGGAACAAGGATGAAGTGA | |
| AGGCATGATTGAGAAAGTCGGGGGGAAGTTGACGGGGA | |
| Pr3-163 Homo sapiens mitochondrial DNA (A point mutation at nt | |
| 169 of the clone) | |
| AGGCTATGTGTTTTGTCAGGGGGTTGAGAATGAGTGTGAGGCGTATT | SEQ ID 52 |
| ATACCATAGCCGCCTAGTTTCAAGAGTACTGCGGCAAGTACTATTGAC | |
| CCAGCGATGGGGGCTTCGACATGGGCTTTAGGGAGTCATAAGTGGAG | |
| TCCGTAAAGAGGTATCTTTAGTATAAAGGCTATTGTGTAAGCTAGTCA | |
| TATTAAGTTGTTGGCTCAGGAGTTTGATAGTTCTTGGGCAGTGAGAGT | |
| GAGTAGTAGAATGTTTAGTGAGCCTAGGGTGTTGTGAGTGTAAATTA | |
| AGTGCGATGAGTAGGGGAAGGGAGCCTACTAGGGTGTAGAATAGGA | |
| AGTATGTCCTGCGTTCAGGCGTTCTGCTGGTTGCCTCATCGGGTGAT | |
| GATAGCCAAGGTGGGGATAAGTGTGGTTCCAAAC | |
| Pr3-165 Homo sapiens ribosomal protein S8 | |
| GAGCGATGGGCATCTGTCGGGACAACTGGCACAAGCGCCGCAAAACC | SEQ ID 53 |
| GGGGGCAAGAGAAAGCCCTACCACAAGAAGCGGAAGTATGAGTTGG | |
| GGCGCCCAGCTGCCAACACCAAGATTGGCCCCCGCCGCATCCACACA | |
| GTCCGTGTGCGGGGAGGTAACAAGAAATACCGTGCCCTGAGGTTGGA | |
| CGTGGGGAATTTCTCCTGGGGCTCAGAGTGTTGTACTCGTAAAACAA | |
| GGATCATCGATGTTGTCTACAATGCATCTAATAACGAGCTGGTTCGTA | |
| CCAAGACCCTGGTGAAGAATTGCATCGTGCTCATCGACAGCACACCG | |
| TACCGACAGTGGTACGAGTCCCACTATGCGCTGCCCTGGGCCGCAAG | |
| AAGGGAGCCAAGCTGACT | |
| Pr3-168 Homo sapiens ubiquitin specific protease 8 (USP) mRNA | |
| GGCACATTGGCTAAAGGCTCTTTGGAGAATGTTTTGGATTCCAAAGA | SEQ ID 54 |
| CAAAACCCAAAAGAGCAATGGTGAAAAGAATGAAAAATGTGAGAGCA | |
| AAGAGAAAGGAGCAATCACAGCAAAGGAACTATACACAATGATGACG | |
| GATAAAAACATCAGCTTGATTATAATGGATGCTCGAAGAATGGAGGA | |
| TTATCAGGATTCCTGTATTTTACATTCTCTCAGTGTTCCTGAAGAAGC | |
| CATCAGTCCAGGAGTCACTGCTAGTTGGATTGAAGCACACCTGCCAG | |
| ATGATTCTAAAGACACATGGAAGAAGAGGGGGNAATGTGGAGTATTG | |
| TGGGTACTTCTTGACTGGGTTTAAGTTCTGCCAAAGATTTACCAGATT | |
| GGAACCAACTCTCCCGGAGTTTGAAAGATGCACTTTTCAGGGGGGAA | |
| AGTAAAACTGGTCCTGCNCATGAGCCTTTGGNTTTAANGGGGGGTTT | |
| GAAACTGGTCCTTTTTNTNCCCCGTTTCCACAAGCTTANGGGCNTCCC | |
| CCNCACCCNANAAAANNGGGNTTCATNGGGTTTNCTTTCCCTTNGGAA | |
| AAAAAATCTTTTAAACGGGGNCCACCCCCCCTTTTTAAAAN | |
| Pr3-170 Homo sapiens sgk protein kinase | |
| TACCNTGTTTGNGCTGGCGCGCCTGCAGGTCGACACTAGTGGATCCAAAG | SEQ ID 55 |
| CAACTCGATTGGCAAGTCCCCTGACAGCGTCCTCGTCACACGCCAGCG | |
| TCAAGGAAGCTGCCGAGGCTTTCCTAGGCTTTTCCTATGCGCCTCCCA | |
| CGGACTCTTTCCTCTGAACCCTGTTAGGGCTTGGTTTTAAAGGATTTT | |
| ATGTGTGTTTCCGAATGTTTTAGTTAGCCTTTTGGTGGAGCCGCCAGC | |
| TGACAGGAGATCTTACAAGAGAATTTGCACATCTCTGGAAGCTTAGCA | |
| ATCTTATTGCACACTGTTCGCTGGAAGCTTTTTGAAGAGCACATTCTC | |
| CTCAGTGAGCTCATGAGGTTTTCATTTTTATTCTTCCTTCCAAGGTGG | |
| TGCTATGTCTGAAACGAGCGTTAAGAGTGCCCGCCTTAGACGGAGCA | |
| NGGAGTTTTCGTTAGAAAAGGGGACGCTGTTCTAAAAAANGTCTCTG | |
| GCAGATCTGTCTGGGCTGGTGATGAGNAATATTATGAAAATGTGNCC | |
| TTTNTGAANAAAATGGGGTTAGCTTCNAACTTTCTTTCGCAAGGGTTC | |
| AAGTTTTTATTTNCCTTGGGAATNCCTGGGGAACCCGCGGGGAAGGG | |
| GGGATGCCNGANCAAAGGNTTTTGTTTAGCGNNAAGGGGACCTTGCG | |
| GACTNCACGGGGAAATTTNTTTGTTT | |
| Pr3-174 Homo sapiens mitochondrial genome | |
| GTGACCAAGACCCTACTTCTAACCTCCCTGTTCTTATGAATTCGAACA | SEQ ID 56 |
| GCATACCCGCGATTGCGCTACGACCAACTCATACACCTCCTATGAAAA | |
| AACTTCCTACCACTCACCCTAGCATTACTrATATGATATGTCTCCATA | |
| CCCATTACAATCTCCAGCATTCCCCCTCAAACCTAA | |
| Pr3-176 Homo sapiens metastasis associated 1 (MTA1) mRNA | |
| GGGACATCTCCAGCACCCTCATCGCCCTGGCCGACAAGCACGCAACC | SEQ ID 57 |
| CTGTCAGTCTGCTATAAGGCCGGACCGGGGGCGGACAACGGCGAGG | |
| AAGGGGAAATAGAAGAGGAAATGGAGAATGCGGAAATGGTGGACCT | |
| GCCCGAGAAACTAAAGCACCAGCTGCGGCATCGGGAGCTGTTCCTCT | |
| CCCGGCAGCTGGAGTCTCTGCCCGCCACGCACATCAGGGGCAAGTGC | |
| AGCGTCACCCTGCTCAACGAGACCGAGTCGCTCAAGTCCTACCTGGA | |
| GCGGGAGGATTTCTTCTTCTATTCTCTAGTCTACGACCCAGAGCAGAA | |
| GACCCTGCTGGCAGATAAAGGAGAGATTCGAGTAGGAAACCGGTACC | |
| AGGCAGACATCACCGACTTGTTAAAAGAAGGCGAGGAGGATGGCCGA | |
| GACCAGTCCAGGTTTGGAGACCCAAGTGTNGGGAGGGGCACAACCCA | |
| CTTACAGACAAGCCAGATGNNCATTCTTGGGNGGNGGGCCGCTTTTG | |
| GGGCACCTTCCACGGGNCCTGGACTGAGANNTTTCTTCCACACCCAC | |
| TTGCAAAGANCNCCNAATTGCTTCCNAAAATANNCCTTTTCCNCCCCT | |
| GGGTTCTTTCAAAANAAATTTACAAATTTCAGGC | |
| Pr3-179 Homo sapiens trans-Golgi p230 mRNA | |
| CCGAGGCGAGCGAGTGGCACCCGGAAGAAAGAGACGCGGCGGGGGC | SEQ ID 58 |
| GACGCCGACACCCTCAGGACGAGTGTCCGGACTTGCCCACAGCCTCA | |
| AGGAGGAGACGGCGAGGCCCGGCCCCCGCTGTCCCTGGTGTAAAGA | |
| AGTCGCCGTAGCCGTCGCGGCCGGGACTCCCCGGGCTCTCGCCCTTC | |
| AGGTTTCGTTGACACTCAGGACGGTACGTACGCTGCGCCATGTTCAA | |
| GAAACTGAAGCAAAAGATCAGCGAGGAGCAGCAGCAGCTCCAGCAG | |
| GCGCTGGCTCCTGCTAGGCGTCCTCCAATTCTTCAACACCAACAAGA | |
| ATGAGGAGCAGGACATCTTCATTTACAGAGCAACTTGATGNAAGGTA | |
| CACCCAATAGAAGAGTTCAAGGTGGACACACAAGTCTTTTGCACAGA | |
| AAGCTTCAGTTCCNGGTGCCCTCGGGGGAGTCTTTGTTTTNGAAGTC | |
| CGATAAGGAATTNTTTTCCGGNCTTTTTTAAAGAGTTTTTTGGTCCAA | |
| AATNTTTCAAAAAATCCTGAATGATTGACTGGAAGNTCTGCCGTTTTG | |
| ATCCCCCTTTTTTGATGNNGGGTAAAAATTGGGGGGGATTTNANACG | |
| NTTAAAAAAAAATGTTTTCNGGTTGNAAAAAAGAANAANN | |
| Pr3-186 Homo sapiens Surf-5 and Surf-6 genes | |
| AGAAAACACAAAAGAAATTCCGGAAGCGAGAAGAGAAGGCTGCTGAG | SEQ ID 59 |
| CACAAGGCCAAGTCCTTGGGGGAGAAATCTCCAGCAGCCTCTGGGGC | |
| CAGGAGGCCTGAGGCAGCCAAAGAGGAAGCAGCTTGGGCTTCCAGCT | |
| CAGCAGGGAACCCTGCAGATGGCCTGGCCACTGAGGCTGAGTCTGTC | |
| TTTGCTCTGGATGTTCTGCGACAGCGACTGCATGAGAAGATCCAGGA | |
| GGCCCGGGGCCAGGGCAGTGCCAAGGAGCTGTCCCCTGCCGCCTTG | |
| GAGAAAAGGCGGCGGAGAAAGCAGGAACGGGACCGGAAGAAGAGGA | |
| AGCGAAAGGAGCTGCGGGCGAAAGANAAGCCAGGAAGGCTGAGGAG | |
| GCCACGGAGGCCCAGGAGGTGGTGGAGGCAACCCCAGAGGGGGCCT | |
| GCACGGACCNCANGAGCCCCCGGCTTGTCTTCATTAGGGGGGAGGTG | |
| AGCGAAACAACCGGCCACAAGGGGCACCAAAAAAAAAAAAGCANAGG | |
| TGAAGGGAACCTCNCCCTNCCGGAGAATACCGCANTTTTGAACCTGC | |
| GCNCCGAAAAGCGTTGANAANTCGCCCNATGGGGGAAGCCCNGACTG | |
| GGCAAANA | |
| Pr3-197 Homo sapiens calcium binding protein (ALG-2) mRNA (6 | |
| nt deletion and a point mutation) | |
| GGTCTCTCGTCGCTGCAGGCGCCTCAGCCCAGCCGCGTGCCTTGGCC | SEQ ID 60 |
| GATGGCCGCCTACTCTTACCGCCCCGGCCCTGGGGCCGGCCCTGGGC | |
| CTGCTGCAGGCGCGGCGCTGCCGGACCAGAGCTTCCTGTGGAACGTT | |
| TTCCAGAGGGTCGATAAAGACAGGAGTGGAGTGATATCAGACACCGA | |
| GCTTCAGCAAGCTCTCTCCAACGGCACGTGGACTCCCTTTAATCCAGT | |
| GACTGTCAGGTCGATCATATCCATGTTTGACCGTGAGAACAAGGCCG | |
| GCGTGAACTTCAGCGAGTTCACGGGTGTGTGGAAGTACATCACGGAC | |
| TGGCAGAACGTCTTCCGCACGTACGACCGGGACAACTCCGGGATGAT | |
| CGATAAGAACGAGCTGAAGCAGGCCCTCTCAGGCTACCGGCTTNTNT | |
| GACCAGTTCCACGACATCCTATTCGAAAAGTTTGACAGGCAGGGACG | |
| GGGCAAAATCGCTTCAACACTTTATCANGGCTGNATTGTGTGAANAG | |
| GTGGCGGTNTTTTAAACTTCACCCGGATAGGANGTGTTTAAGGGGTC | |
| ACNAAAAANCTGCCANGNTTAAAANTCGAAGACNGCCCCTTGGGAG | |
| GGCCCCACTNGGAAGGCCAATGTNCCNT | |
| Pr3-200 Mus musculus BS4 peptide mRNA | |
| GCGCAGGGATGGCACAAAAGAAATATCTTCAAGCAAAATTGACCCAG | SEQ ID 61 |
| TTTTTAAGGGAAGACAGGATTCAACTTTGGAAACCTCCATATACAGAT | |
| GAAAATAAAAAAGTTGGTTTGGCATTAAAGGACCTTGCTAAGCAGTA | |
| CTCTGACAGACTAGAATGCTGTGAAAATGAAGTAGAAAAGGTAATAG | |
| AAGAAATACGTTGCAAGGCAATTGAGCGTGGAACAGGAAATGACAAT | |
| TATAGAACAACGGGAATTGCTACAATCGAGGTGTTTTTACCACCAAGA | |
| CTAAAAAAAGATAGGAAAAACTTGTTGGAGACCCGATTGCACATCAC | |
| TGGCAGAGAACTGAGGTCCAAAATAGCTGAAACCTTTGGACTTCAAG | |
| AAAATTATATCAAAATTGTCATAAATAAGAAGCAACTACACTAGGGAA | |
| AACCCTTGAAGAAAAGGCGTGGCTCCAATGTGAAAGCGATGGTGCTT | |
| GACTAAAACATCTGAAAGGACGCAGGAAACTTCCGTTGGGGAAGAGA | |
| GCAAAANAGGCCACTCAAGAAAACANTCGNGNCAGANGGCTTGAATC | |
| TGGCAGAAGCACNAAAGNGGGGGACCAAAGAACCCNCTTTAACTTNT | |
| TACNGNCGGCNATN | |
| Pr3-203 Homo sapiens Pig11 (P1011 mRNA) | |
| GGCTCTGGCACACAGCTGTGCTCACAAAATACTGGGTGGCTTGGTTA | SEQ ID 62 |
| GAGCTAATTGTAGTGGAGCCTGCAGGTGAGGGTGAGGGAGGGGGCT | |
| GCAGGTCAGGTAAGATCTGGAAGACAGACGTCAGCTTGGAGGGCAG | |
| GGGGAGTCTAAGGCAAGGAGATTTACAGTTGGGAAGGAGGCAGTGG | |
| CAGAGGGGTGAGGGACAGGGGCCCTTAAGTCCAGCGAGGAAAGCTC | |
| GGTGTGGGCCCGCTCTACGCTCCGTTTGGGGTGACCTGGAACGCCTC | |
| TTCTCCCAGCTCCCCTCCAGCCATCAGCAGCCTCTTGTCAAGCTTCTGC | |
| CTCGCCCCAGTCTATCCCCAACCCCAAATCAAGACGACCTTTCTTCAC | |
| GGTCACTATTTATTCTTTGGTCCTTTTCTTTTTGTAAGAAACATTCACA | |
| AAAACCAGTGCCNNNCCCCNNNNNNNNNNNNNNNNNNNNNNNNNNNNN | |
| NNNNNNNNNNNNNNNAAAAAGTCGGGAGTCTTTTAAGGGGGCGNGGC | |
| CNTNGNTTTCCCCGGGGGGGCCCGGNAAAGGNCCCCATNCCTTTNGG | |
| GGGGGGGTTNNATNTGGGCCCGGNTTAAAACNTNGATNGNACCNCTG | |
| GCT | |
| Pr3-206 Homo sapiens FIFO-type ATP synthase sub-unit d mRNA | |
| GCAGCCAGGGTCGGTGAAGGATCCCAAAATGGCTGGGCGAAAACTTG | SEQ ID 63 |
| CTCTAAAAACCATTGACTGGGTAGCTTTTGCAGAGATCATACCCCAGA | |
| ACCAAAAGGCCATTGCTAGTTCCCTGAAATCCTGGAATGAGACCCTC | |
| ACCTCCAGGTTGGCTGCTTTACCTGAGAATCCACCAGCTATCGACTG | |
| GGCTTACTACAAGGCCAATGTGGCCAAGGCTGGCTTGGTGGATGACT | |
| TTGAGAAGAAGTTTAATGCGCTGAAGGTTCCCGTGCCAGAGGATAAA | |
| TATACTGCCCAGGTGGATGCCGAAGAAAAAGAAGATGTGAAATCTTG | |
| TGCTGAGTGGGGTGTCTCTCTCAAAGGCCAGGATTGTAGAATATGAA | |
| GAAAGAGATGGAGAAGATGAAAGAACTTAATTNCTTTTGATCAGATG | |
| ACCATTGANGGACTTGAATGAAGCTTTTCCAGAAACCAAATTAGACAA | |
| GAAAAAGTNTCCTATTGGNCTCACCANCCATTGGGAATTATAAAATGA | |
| GTCNGGAGGAAGTTTGGCCTTGNTACCATTTGGCCTTAAATATTATTT | |
| TCCCNNNNNNNNNNNNNNNNNNNNNNNNNNNNNAAAAAACCTCGGGGN | |
| CTT | |
| Pr3-209 Homo sapiens ribosomal protein L18a | |
| GCTGTCAAGCAGTTCCACGACTCCAAGATCAAGTTCCCGCTGCCCCA | SEQ ID 64 |
| CCGGGTCCTGCGCCGTCAGCACAAGCCACGCTTCACCACCAAGAGGC | |
| CCAACACCTTCTTCTAGGTGCAGGGCCCTCGTCCGGGTGTGCCCCAA | |
| ATAAACTCAGGAACGCCCCAAAAAAAAAAAAAAAAAAAAAAAAAAAA | |
| AAAAAAAAAAAAAAAAAAAAAAAAAAAAAAAAAAAAAAAAAAAAAAA | |
| AAAAAAAAC | |
| Pr3-219 Human FACL5 for fatty acid coenzyme A ligase 5 | |
| GTTGCTGCTTCTCAGATGCCAAGACTATGTATGAGGTTTTCCAAAGAG | SEQ ID 65 |
| GACTCGCTGTGTCTGACAATGGGCCCTGCTTGGGATATAGAAAACCA | |
| AACCAGCCCTACAGATGGCTATCTTACAAACAGGTGTCTGATAGAGC | |
| AGAGTACCTGGGTTCCTGTCTCTTGCATAAAGGTTATAAATCATCACC | |
| AGACCAGTTTGTCGGCATCTTTGCTCAGAATAGGCCAGAGTGGATCA | |
| TCTCCGAATTGGCTTGTTACACCGTACTCTATGGTAGCTTGTACCTCT | |
| GTATGACACCTTGGGACCAGAAGCCATCGTACATATTGTCAACAAGG | |
| CTGATATCGCCGTGGTGATCTGTGACACACCCCAAAAGGCATTGGTG | |
| CTGATAGGGAATGTAAGAAGGCTCACCC | |
| Pr3-224 Homo sapiens DNA-binding protein (HRC1) mRNA (The | |
| clone contains alternative exon la;it might be a new isoform | |
| of HRC1) | |
| CCGGATNGGGTCTGCAGGCTGGCGAGCGCCCAGGCCAGACTGGCCG | SEQ ID 66 |
| GTTTGTGGTTGTGCAGCGGCTTCGGGAGAAGGAGCGGCAGTTGCTGC | |
| CACAAGAGTGTCCAGTGGGCGCCCAGGCCACCCTGCGGACAGTTTGC | |
| CAGCGATGTCCAGTTTGTCCTGAGGCGCACAGGGCCCAGCCTAGCTG | |
| GGAGGCCCTCCTCAGACAGCTGTCCACCCCCGGAACGCTGCCTAATT | |
| CGTGCCAGCCTCCCTGTAAAGCCACGGGCTGCGCTGGGCTGTGAGCC | |
| CCGCAAAACACTGACCCCCGAGCCAGCCCCCAGCCTCTCAGGCCCTG | |
| GGCCTGCGGGCCCTGTGACACCCACACCAGGCTGCTGCACAGACCTG | |
| CGGGCCTGAACTCAGGGTGCAGAGGAC | |

1. The use of an isolated nucleic acid molecule comprising a sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 to detect or monitor cancer.

2. The use of a nucleic acid probe which is capable of hybridising under high stringency conditions to an isolated nucleic acid molecule comprising a sequence selected from SEQ.ID. 1, SEQ.ID. 2, SEQ.ID. 3, SEQ.ID. 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 to detect or monitor cancer.

3. A method of detecting or monitoring cancer comprising the step of detecting or monitoring elevated levels of a nucleic acid molecule comprising a sequence selected from SEQ.ID. 1, SEQ.ID. 2, SEQ.ID. 3, SEQ.ID. 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 in a sample from a patient.

4. A method of detecting or monitoring cancer comprising the use of a nucleic acid molecule or probe according to claim 1 or claim 2 in combination with a reverse transcription polymerase chain reaction (RT-PCR).

5. A method of detecting or monitoring cancer comprising detecting or monitoring elevated levels of a protein or peptide comprising an amino acid sequence encoded by a nucleic acid sequence selected from SEQ.ID. 1, SEQ.ID. 2, SEQ.ID. 3, SEQ.ID. 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66.

6. A method according to claim 5 comprising the use of an antibody selective for a protein or peptide as defined in claim 5 to detect the protein or peptide.

7. A method according to claim 7 comprising the use of an Enzyme-link led Immunosorbant Assay (ELISA).

8. Use or method according to any one of claims 1 to 7, wherein the cancer is prostate cancer is prostate cancer.

9. A kit for use with a method according to any one of claims 3-8 comprising a nucleic acid, protein or peptide, or an antibody as defined in any one of claims 3-8.

10. A method of prophylaxis or treatment of cancer comprising administering to a patient a pharmaceutically effective amount of nucleic acid molecule comprising a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 or a pharmaceutically effective fragment thereof.

11. A method of prophylaxis or treatment of cancer comprising administering to a patient a pharmaceutically effective amount of a nucleic acid molecule hybridisable under high stringency conditions to a nucleic acid molecule comprising a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID: 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 or a pharmaceutically effective fragment thereof.

12. A method of prophylaxis or treatment of cancer comprising administering to a patient a pharmaceutically effective amount of a protein or peptide comprising an amino acid sequence encoded by a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 or a pharmaceutically effective fragment thereof.

13. A method of prophylaxis or treatment of cancer comprising the step of administering to a patient a pharmaceutically effective amount of an antibody capable of specifically binding a protein or peptide comprising an amino acid sequence encoded by a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66.

14. A method according to any one of claims 10 to 11, wherein the cancer is prostate cancer.

15. A vaccine comprising a nucleic acid molecule having a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID.10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 or a pharmaceutically effective fragment thereof and a pharmaceutically acceptable carrier.

16. A vaccine comprising a protein or peptide comprising an amino acid sequence encoded by a nucleic acid sequence selected from SEQ.ID. 1, SEQ.ID. 2, SEQ.ID 3, SEQ.ID 4, SEQ.ID. 5, SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 31, SEQ.ID. 32, SEQ.ID. 33, SEQ.ID. 34, SEQ.ID. 35, SEQ.ID. 36, SEQ.ID. 37, SEQ.ID. 38, SEQ.ID. 39, SEQ.ID. 40, SEQ.ID. 41, SEQ.ID. 42, SEQ.ID. 43, SEQ.ID. 44, SEQ.ID. 45, SEQ.ID. 46, SEQ.ID. 47, SEQ.ID. 48, SEQ.ID. 49, SEQ.ID. 50, SEQ.ID. 51, SEQ.ID. 52, SEQ.ID. 53, SEQ.ID. 54, SEQ.ID. 55, SEQ.ID. 56, SEQ.ID. 57, SEQ.ID. 58, SEQ.ID. 59, SEQ.ID. 60, SEQ.ID. 61, SEQ.ID. 62, SEQ.ID. 63, SEQ.ID. 64, SEQ.ID. 65 and SEQ.ID. 66 or a pharmaceutically effective fragment thereof, and a pharmaceutically acceptable carrier.

17. An isolated mammalian nucleic acid molecule comprising a nucleic acid sequence selected from SEQ.ID. 6, SEQ.ID. 7, SEQ.ID. 8, SEQ.ID. 9, SEQ.ID. 10, SEQ.ID. 11, SEQ.ID. 12, SEQ.ID. 13, SEQ.ID. 14, SEQ.ID. 15, SEQ.ID. 16, SEQ.ID. 17, SEQ.ID. 18, SEQ.ID. 19, SEQ.ID. 20, SEQ.ID. 21, SEQ.ID. 22, SEQ.ID. 23, SEQ.ID. 24, SEQ.ID. 25, SEQ.ID. 26, SEQ.ID. 27, SEQ.ID. 28, SEQ.ID. 29, SEQ.ID. 30, SEQ.ID. 43, SEQ.ID. 44, SEQ.ID. 52, SEQ.ID. 60 and SEQ.ID. 66 or a variant of a fragment thereof which encodes a prostate-associated antigen which is expressed in higher than normal concentrations in prostate cancer cells.

18. A vector comprising an isolated mammalian nucleic acid molecule according to claim 17.

19. A nucleic acid molecule comprising at least 15 nucleotides, the nucleic acid molecule being capable of hybridising to a molecule according to claim 17 under high stringency conditions.

20. An isolated protein or peptide comprising an amino acid sequence obtainable from a nucleic acid molecule according to claim 17, 18 or 19.

21. A nucleic acid probe capable of hybridising to a nucleic sequence as defined in SEQ ID 34, SEQ ID 35, SEQ ID 43, SEQ ID 44, SEQ ID 52, SEQ ID 60, SEQ ID 65 or SEQ ID 66, or a sequence complementary thereto, under high stringency conditions.
